(19) United States
(12) Patent Application Publication
(10) Pub. No.: US 2023/0389909 A1
(43) Pub. Date: Dec. 7, 2023

(54) CATHETER LOCKING MECHANISMS AND METHODS FOR SAME

(71) Applicants: Alex Marine, Eden Prairie, MN (US); Karl V. Ganske, Hopkins, MN (US); Steve Michael, Eden Prairie, MN (US)

(72) Inventors: Alex Marine, Eden Prairie, MN (US); Karl V. Ganske, Hopkins, MN (US); Steve Michael, Eden Prairie, MN (US)

(21) Appl. No.: 18/234,835

(22) Filed: Aug. 16, 2023

Related U.S. Application Data
(63) Continuation-in-part of application No. 17/896,589, filed on Aug. 26, 2022.
(60) Provisional application No. 63/237,958, filed on Aug. 27, 2021.
(60) Provisional application No. 63/348,128, filed on Jun. 2, 2022.

(51) Int. Cl. A61B 17/00; A61F 2/01

(52) U.S. Cl. CPC A61B 17/00234; A61M 2025/0006; A61F 2/011; A61F 2/013

(57) ABSTRACT

A locking catheter system includes a locking mechanism. The locking mechanism includes a lock body having a system lumen extending through the lock body, wherein first and second catheters are received in the system lumen. A system channel extends from the system lumen, and the system channel is configured to receive one or more of the first or second catheters. A locking channel having a lock element extends from the system lumen. The locking channel is configured to receive one or more of the first or second catheters. In an unlocked configuration the first and second catheters are received in the system channel and are movable relative to the lock body. In the locked configuration at least the second catheter is received in the locking channel, the second catheter is statically coupled with the lock body, and the first catheter is movable relative to the lock body.

### PRIORITY APPLICATIONS

This application is a continuation-in-part of U.S. patent application Ser. No. 17/896,589, filed Aug. 26, 2022, which application claims the benefit of priority to both U.S. Provisional Application Ser. No. 63/348,128, filed Jun. 2, 2022 and U.S. Provisional Application Ser. No. 63/237,958, filed Aug. 27, 2021, all of which are incorporated herein by reference in their entireties.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings that form a part of this document: Copyright Surmodics, Inc. of Eden Prairie, MN, USA. All Rights Reserved.

### TECHNICAL FIELD

This document pertains generally, but not by way of limitation, to catheters.

### BACKGROUND

Mechanical thrombectomy procedures can involve deployment of a capture device at thrombus. The capture device is navigated past the thrombus in a collapsed configuration before deployment. In one example, the capture device is navigated along another instrument, such a procedural guidewire, sometimes referred to as an over the wire (OTW) approach. The procedural guidewire is previously navigated to a location proximate to the thrombus, and the capture device (e.g., coupled with its own catheter) is navigated to the location along the procedural guidewire. In one example, a technician holds a proximal end of the procedural guidewire to minimize movement of the guidewire potentially caused through navigation of the capture device and its catheter along the guidewire.

Once deployed, the capture device is proximally pulled through the thrombus relative to the procedural guidewire to mechanically capture or entrain the thrombus for removal through a separate retrieval catheter or sheath. Optionally, the capture device is then collapsed, re-navigated along the procedural guidewire past the thrombus and redeployed. Once deployed again, the capture device is proximally pulled to capture additional thrombus. This process of capture, retraction to the retrieval catheter and re-navigation (both) along the procedural guidewire, and re-deployment are repeated until the thrombus is removed.

Each of the components, the procedural guidewire, the capture device and its associated catheter, the retrieval catheter or the like are, in some examples, nested within each other and moveable relative to each other.

### Overview

The present inventors have recognized, among other things, that a problem to be solved can include minimizing (decreasing or eliminating) unspecified movement of components of catheter systems caused through specified movement of other components of the catheter systems. In an example, a thrombectomy procedure includes multiple separate instruments (e.g., catheters) including, but not limited to, a procedural guidewire, basket catheter, basket sheath catheter, and retrieval catheter (e.g., an aspiration catheter, trumpet catheter or the like). These instruments are navigated to a specified location in the vasculature having thrombus starting with a first catheter, such as a procedural guidewire. The procedural guidewire is navigated across the thrombus and provides a rail for the delivery of instruments across the thrombus. A second catheter, for instance a basket catheter having one or more deployable baskets, is navigated along the procedural guidewire to the specified location. In one example, the basket catheter includes a basket sheath catheter that covers the deployable baskets and maintains the baskets in a stowed configuration.

A retrieval catheter is navigated over the basket catheter while the procedural guidewire remains in place. During navigation, the retrieval catheter is first fed distally over the procedural guidewire, for instance a proximal end of the procedural guidewire outside of the patient. Further distal movement of the retrieval catheter is conducted over the basket catheter (also positioned along the procedural guidewire). The retrieval catheter is navigated along the basket catheter and procedural guidewire toward the thrombus. During navigation there is frictional engagement between the retrieval catheter and the basket catheter. Until a proximal end of the retrieval catheter is navigated past a proximal end of the basket catheter the basket catheter is not held and accordingly is not anchored. Instead, it is coupled between the procedural guidewire and the retrieval catheter and ‘floats’. The procedural guidewire is held and the retrieval catheter is held (and manipulated by the clinician), however, the basket catheter is interposed between the guidewire and retrieval catheter and is not held. Accordingly, engagement between the retrieval catheter inner wall and the basket catheter outer wall (e.g., a basket catheter sheath) and friction therebetween imparts proximal force form the retrieval catheter to the basket catheter. The proximal force, in some examples, moves the basket catheter distally in an uncontrolled manner. For instance, because the basket catheter ‘floats’ between the procedural guidewire and the retrieval catheter the basket catheter is readily pushed through engagement and friction between the distally navigated retrieval catheter and the basket catheter. This unspecified movement in some examples includes movement of the basket catheter, such as the associated deployable baskets, away from thrombus and into healthy vasculature.

In other examples, the procedural guidewire includes a lubricious coating to facilitate sliding movement of catheters over the procedural guidewire. For instance, the basket catheter during its initial navigation is readily slid over the procedural guidewire and past the thrombus. However, the lubricious coating may, in some examples, decrease friction between the procedural guidewire and the basket catheter. Accordingly, the proximal force imparted from the retrieval catheter to the basket catheter prompts additional unspecified movement of the basket catheter because of the lubricious coating, thereby further moving the basket catheter away from thrombus and into healthy vasculature.

The present subject matter can help provide a solution to this problem, such as with a locking catheter system having a locking mechanism that secures an otherwise ‘floating’ catheter to another catheter that is itself retained in a specified position. In one example, a first catheter (e.g., a procedural guidewire) is received within a lumen of a second catheter, such as the basket catheter. A locking mechanism is coupled with the basket catheter, for instance proximate to a proximal end. The locking mechanism includes a locking collet and an actuator collar movably coupled along the locking collet. The locking mechanism has a profile approximating (e.g., matching or nearly matching) the profile of the second catheter (the basket catheter in this example) to permit slidable movement of catheters over the locking mechanism and the basket catheter.

As provided in the example above, the procedural guidewire is navigated to thrombus and past the thrombus. The basket catheter with the baskets and the locking mechanism is navigated along the procedural guidewire, with the guidewire received in a lumen of the basket catheter. The baskets of the basket catheter are navigated distal to the thrombus (e.g., a specified location for the baskets). Optionally, the baskets are deployed, for instance by retraction of a basket sheath.

With the procedural guidewire and the basket catheter at specified locations the locking mechanism is transitioned to a locked configuration. For instance, the actuator collar is moved relative to the locking collet. One or more actuator bosses of the actuator collar are engaged with one or more locking struts of the locking collet, and the locking struts are deflected by the engagement. The locking struts are, in one example, deflected into the lumen of the basket catheter (e.g., the lumen of the locking collet in this example). The deflected locking struts engage with the procedural guidewire, and the basket catheter is secured to the procedural guidewire. In one example, the locking struts are positioned on the locking collet relative to the actuator bosses of the actuator collar to ensure one or more of the struts and bosses align with movement of the actuator collar into the locked configuration. Accordingly, the clinician does not have to feel or search for alignment for the locked configuration. One example of this positioning includes the locking struts and actuator bosses staggered around the respective locking collet and actuator collar with coincident profiles to ensure alignment of one or more of the bosses and struts with movement to the locked configuration. For instance, in an example coincident profile the locking struts are staggered in first positions and the actuator bosses are staggered at second positions (e.g., a interval, spacing or the like) different from the first positions to ensure one or more of the bosses aligns with one or more of the locking struts in the locked configuration. In another example of a coincident profile the locking struts and actuator bosses are positioned with similar patterns (e.g., matching, identical, or partially matching) on the respective locking collet and actuator collar with frequency to ensure one or more locking struts align with one or more actuator bosses in the locked configuration.

The retrieval catheter is navigated over the procedural guidewire and the basket catheter secured to the guidewire. Because the locking mechanism has a profile similar to that of the basket catheter the retrieval catheter is readily moved over the locking mechanism and the basket catheter. The procedural guidewire is grasped by a clinician as the retrieval catheter is navigated distally over the guidewire. Because the basket catheter is secured to the procedural guidewire with the locking mechanism the basket catheter does not ‘float’, and movement of the retrieval catheter (and friction between it and the basket catheter) does not cause unspecified movement of the basket catheter. Instead, the basket catheter is readily held in place with the procedural guidewire.

The retrieval catheter is navigated over the basket catheter to a specified location proximal to the thrombus, and initial navigation to the thrombus is completed. In this position, a proximal end of the retrieval catheter is distal to the locking mechanism thereby revealing the actuator collar for manipulation. The actuator collar is moved relative to the locking collet to an unlocked configuration to disengage the one or more actuator bosses of the collar from the locking struts of the collet. The locking struts relax from the procedural guidewire, and relative movement between the basket catheter and the procedural guidewire is permitted. For instance, the basket catheter having deployed baskets is retracted proximally relative to the procedural guidewire, captures thrombus, and pulls the captured thrombus into the retrieval catheter. If movement of the retrieval catheter is specified, and movement of the basket catheter is not specified, the locking mechanism is readily transitioned again to the locked configuration to minimize movement of the basket catheter otherwise caused by movement of the retrieval catheter.

The present inventors have recognized, among other things, that another problem to be solved can include selectively coupling and decoupling the movements of separate components of catheter systems. As noted herein, a thrombectomy procedure includes multiple separate instruments (e.g., catheters) including, but not limited to, a procedural guidewire, basket catheter, basket sheath catheter, and retrieval catheter (e.g., an aspiration catheter, trumpet catheter or the like). During the procedure, for instance during one or more of navigation, manipulation proximate to the thrombus, or during withdrawal of components from the vasculature cooperative movement of components at the same time and in the same direction (e.g., proximally or distally) is specified. Similarly, during the procedure decoupled movement of components relative to each other, whether proximal and distal, proximal and static, distal and static or the like is specified. In still other examples, combinations coupled and decoupled movement are specified.

In one example, a procedure is completed and retraction of a portion of the catheter system, such as a basket catheter, basket sheath catheter, and retrieval catheter is specified while additional procedures may be conducted with the procedural guidewire. Accordingly, maintaining the procedural guidewire stationary (e.g., and positioned proximate to the thrombus or vascular location) is specified. It may be difficult to statically couple a subset of instruments together for retraction while leaving one or more instruments decoupled from the subset and stationary relative to retraction. Instruments have narrow profiles to fit within vasculature, and identification of multiple narrow profile instruments is difficult, holding of one or more of those instruments static while withdrawing others in a coordinated manner is also difficult.

Conversely, in other examples the same one or more instruments that may be specified for decoupling (e.g., to permit relative movement) may, in other circumstances, be specified for static coupling to permit consolidated movement with the remainder of the catheter system. In one example, the catheter system including the basket catheter, basket sheath catheter and the retrieval catheter are moved together in a consolidated manner, for instance along the procedural guidewire. Accordingly, static coupling between one or more of the basket catheter, basket sheath catheter and the retrieval catheter is specified. In another example, movement of one or more components relative to others (previously statically coupled) is specified. For instance, with the basket catheter and basket sheath catheter positioned at a specified location proximate thrombus the clinician navigates the retrieval catheter along basket catheter, basket sheath catheter and the procedural guidewire nested within the basket catheter. The toggling of static coupling between components of the catheter system to selectively permit relative movement and consolidated movement of the components is thereby specified.

The present subject matter can help provide a solution to this problem, such as with a locking catheter system having a locking mechanism that secures various catheter system components together with a static coupling for consolidated movement and that permits decoupling of various catheter system components to permit relative movement between the catheter system components. The locking mechanism is coupled with one or more catheters, for instance first and second catheters, such as a procedural guidewire, a basket catheter or the like. In one example, the locking mechanism is coupled with a retrieval catheter including a deployable mesh trumpet or the like that receives one or more baskets of the basket catheter, and cooperatively captures and compresses thrombus nested within the baskets and trumpet. In one example, the locking mechanism includes a lock body, and the lock body is coupled with a handle assembly of the retrieval catheter. Accordingly, locking of one of the catheters, such as the basket catheter with the locking mechanism permits consolidated movement of the retrieval catheter and the basket catheters (e.g., for withdrawal from vasculature) together while the procedural guidewire remains movably coupled. In this configuration, the locking mechanism permits the sliding movement of the retrieval catheter and basket the catheter over the procedural guidewire, and the procedural guidewire thereby remains in place.

The locking mechanism in this example includes a lock body having a system lumen (e.g., a passage or the like). The system lumen receives one or more catheters, such as a first or second catheter, and in one example a first catheter such as a procedural guidewire and a second catheter such as a basket catheter. The first and second catheters are movably coupled in the system lumen.

The locking mechanism further includes a system channel and a locking channel that are each in communication with the system lumen. The locking channel includes a locking element configured to statically couple either or both of the first or second catheters selectively positioned in the locking channel, as described herein. The locking element includes, but is not limited to, a wedge brake, one or more cleats, one or more biasing elements (e.g., spring biased detent, magnet or the like), collet, set screw or the like.

In contrast to the system lumen, the system channel and the locking channel are accessible laterally. For example, the system channel includes a system access cleft extending along the system channel and the locking channel includes a lock access cleft extending along the locking channel. The system and lock access clefts extend through the lock body provide open access to the respective system and locking channels. As described herein, the clefts permit selective movement and reception of instruments, such as the first or second catheters, between the system channel and the locking channel.

In operation the first and second catheters extend through the system lumen and continue on through the system channel. In this configuration each of the first and second catheters are unlocked and movable relative to the lock mechanism. In an example including a retrieval catheter coupled with the lock mechanism the first and second catheters are movable relative to the retrieval catheter and conversely the retrieval catheter is movable relative to the first and second catheters.

When transition to the locked configuration is specified, for instance to withdraw the retrieval catheter, basket catheter and thrombus together, while leaving the procedural guidewire in place, the lock mechanism is operated. In this example, a second catheter portion of the second catheter (e.g., basket catheter) is grasped by the clinician. The second catheter portion is moved out of the system channel, for instance through the system access cleft that provides lateral access to the portion and permits lateral movement of the portion. The second catheter portion is moved into the locking channel, for instance through a lock access cleft that provides later access to the locking channel. In an example, movement of the second catheter portion is a pivoting movement between the system channel and the locking channel. For instance, the second catheter portion pivots at an access intersection for the system lumen, the system channel, and the locking channel. Because the remainder of the second catheter is retained in the system lumen (e.g., an enclosed lumen) the second catheter portion is readily pivotable between the system and locking channels.

With the second catheter positioned in the locking channel the lock element statically couples the second catheter to the locking mechanism (e.g., the lock body). With the locking mechanism coupled with a retrieval catheter or other instruments, for instance with a handle assembly, movement of the retrieval catheter is transmitted to the locked second catheter. Accordingly, the retrieval catheter and the second catheter are movable cooperatively. Conversely, the first catheter (e.g., a procedural guidewire) remains in the system channel and is movable relative to the retrieval catheter and the locked second catheter. The second catheter and the retrieval catheter are thereby readily withdrawn over the first catheter in the manner of a shuttle traveling on a rail (the first catheter). The first catheter, such as a procedural guidewire, remains in place for additional procedural use.

In another example, both of the first and second catheters are moved to the locking channel and are engaged by one or more locking elements. In this locked configuration both of the first and second catheters are statically coupled with the locking mechanism. Retraction of the locking mechanism (in an example coupled with a retrieval catheter) also retracts both of the first and second catheters statically coupled with the locking mechanism. Accordingly, the clinician selectively positions one or more catheters in the locking channel and the system channel to vary static coupling and movable coupling of various catheters in combinations specified by the clinician.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a retrieval system.

FIG. 2 is a schematic side view illustrating the locating of a positioning guidewire across a clot within a blood vessel according to an aspect of the present disclosure.

FIG. 3 is a schematic side view illustrating the positioning of a capture/delivery catheter within the blood vessel according to an aspect of the present disclosure.

FIG. 4 is a schematic side view illustrating the positioning of a filter delivery catheter within the blood vessel according to an aspect of the present disclosure.

FIG. 5 is a schematic view illustrating the filter delivery catheter shown in FIG. 4 with the positioning guidewire removed from the blood vessel.

FIG. 6 is a schematic view illustrating the deployment of a filter on a filter guidewire according to an aspect of the present disclosure.

FIG. 7 is a schematic view illustrating the positioning of a capture sleeve sheath according to an aspect of the present disclosure.

FIG. 8 is a schematic view illustrating the deployment of a capture sleeve according to an aspect of the present disclosure.

FIG. 9 is a side view of a retrieval system according to an aspect of the present disclosure.

FIG. 10 is a partial side view of a filter assembly and a capture sleeve of the retrieval system depicted in FIG. 9.

FIG. 11 is a side view of a filter catheter received within a filter delivery catheter of the retrieval system depicted in FIG. 9.

FIG. 12 is a partial side view of the filter catheter depicted in FIG. 11.

FIG. 13 is a cross sectional view of the filter catheter of the retrieval system depicted in FIG. 11.

FIG. 14 is a partial side view of a filter assembly of the filter catheter depicted in FIG. 13.

FIG. 15 is a partial cross-sectional side view of an atraumatic tip of the filter catheter according to an aspect of the present disclosure.

FIG. 16 is a schematic side view illustrating the locating of a positioning guidewire across a clot within a blood vessel according to an aspect of the present disclosure.

FIG. 17 is a schematic side view illustrating the positioning of a capture/delivery catheter within the blood vessel according to an aspect of the present disclosure.

FIG. 18 is a schematic side view illustrating the positioning of a filter delivery catheter within the blood vessel according to an aspect of the present disclosure.

FIG. 19 is a schematic side view illustrating the deployment of at least one filter on a filter catheter according to an aspect of the present disclosure.

FIG. 20 is a schematic side view illustrating the deployed filter catheter depicted in FIG. 19 with the filter delivery catheter pulled axially to expose the filters according to an aspect of the present disclosure.

FIG. 21 is a schematic side view illustrating the positioning of a capture sleeve sheath according to an aspect of the present disclosure.

FIG. 22 is a schematic side view illustrating the deployment of a capture sleeve according to an aspect of the present disclosure.

FIG. 23 is a schematic side view illustrating a retrieval system and an aspiration sheath according to an aspect of the present disclosure.

FIG. 24 is a side view of an aspiration sheath according to an aspect of the present disclosure.

FIG. 25A is a schematic view of a structural layer of an aspiration sheath according to an aspect of the present disclosure.

FIG. 25B is a schematic view of a structural layer of an aspiration sheath according to an aspect of the present disclosure.

FIG. 26 is a partial cross-sectional side view of an aspiration sheath with a dilator according to an aspect of the present disclosure.

FIG. 27A is a schematic side view illustrating a collection catheter and basket catheter retrieving thrombus.

FIG. 27B is a schematic side view of the collection and basket catheters of FIG. 27A with the collection catheter plugged with thrombus.

FIG. 28 is a schematic side view of one example of a thrombus capture and conveyor system.

FIG. 29A is a schematic side view of the thrombus capture and conveyor system of FIG. 28 having a thrombus conveyor in a full everted configuration at deployment.

FIG. 29B is a schematic side view of the thrombus capture and conveyor system of FIG. 28 having the thrombus conveyor in an everted configuration including partial inversion at or after deployment.

FIG. 30 is a detailed side schematic view of the thrombus capture and conveyor system of FIG. 28 illustrating a conveyor mouth profile of the thrombus conveyor.

FIG. 31A is a schematic side view of the thrombus capture and conveyor system of FIG. 28 with thrombus between a collection basket and the thrombus conveyor.

FIG. 31B is a schematic side view of the thrombus capture and conveyor system of FIG. 28 with thrombus captured between the collection basket and the thrombus conveyor.

FIG. 31C is a schematic side view of the thrombus capture and conveyor system of FIG. 28 with the thrombus conveyor in the process of inverting and shuttling thrombus toward a conveyor sheath.

FIG. 31D is a schematic side view of the thrombus capture and conveyor system of FIG. 28 with the thrombus conveyor enveloping the thrombus and the collection basket, and shuttling thrombus toward the conveyor sheath.

FIG. 32A is a schematic side view of another example of a thrombus capture and conveyor system with thrombus between multiple collection baskets and the thrombus conveyor.

FIG. 32B is a schematic side view of the thrombus capture and conveyor system of FIG. 32A with thrombus captured between the collection baskets and the thrombus conveyor.

FIG. 32B′ is a schematic side view of the thrombus capture and conveyor system of FIG. 32A with thrombus separated with the collection baskets.

FIG. 32C is a schematic side view of the thrombus capture and conveyor system of FIG. 32A with the thrombus conveyor in the process of inverting and shuttling thrombus toward the conveyor sheath.

FIG. 33 is a perspective view of an example basket catheter, trumpet catheter and trumpet sheath.

FIG. 34 is a side view of one example of catheter system.

FIG. 35 is a side view of one example of a locking mechanism as part of the catheter system of FIG. 34.

FIG. 36 is a side view of the locking mechanism of FIG. 35.

FIG. 37A is a schematic view of an example locking collet of the locking mechanism of FIG. 36.

FIG. 37B is a first sectional view of the locking mechanism of FIG. 36 including an example actuator boss and an example locking strut.

FIG. 37C is a second sectional view of the actuator boss deflecting the locking strut of FIG. 37B.

FIG. 38A is a first sectional view of an example collet detent and an example collar shoulder of the locking mechanism of FIG. 36.

FIG. 38B is a second sectional view of the collet detent engaged with the collar shoulder of FIG. 38A.

FIG. 39 is a perspective view of one example of a catheter system including a handle assembly and a selective locking mechanism.

FIG. 40A is a top view of one example of a selective locking mechanism including system and lock access clefts.

FIG. 40B is a sectional view of one example of a selective locking mechanism including a biasing lock element.

FIG. 40C is a top view of one example of a selective locking mechanism including a cleat lock element.

FIG. 41 is a block diagram of one example of a method for locking catheters.

### DETAILED DESCRIPTION

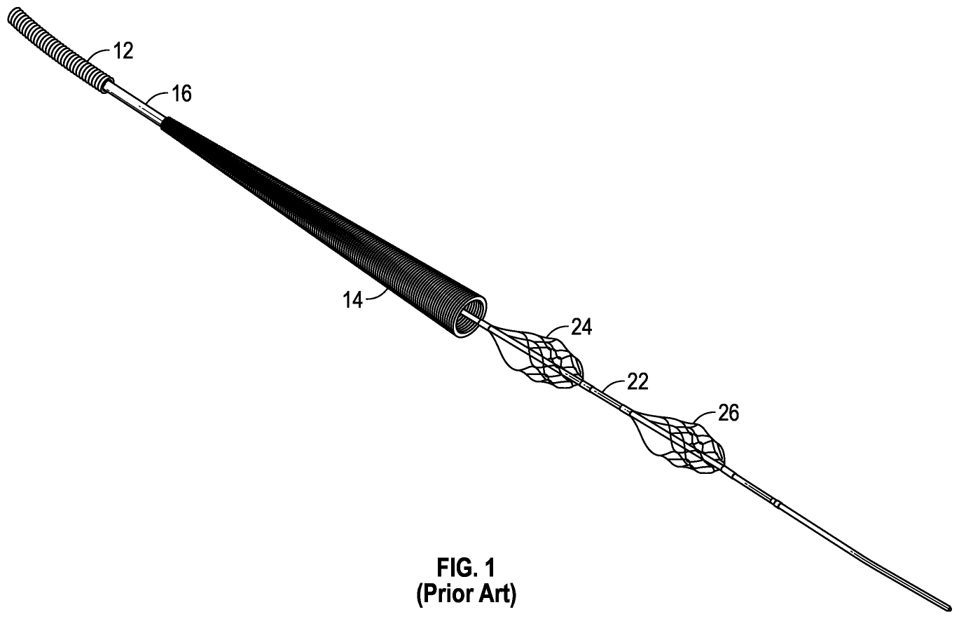

As shown in FIG. 1, the retrieval system 10 can comprise (i) a flexible capture sleeve 14 on the distal end of a flexible sleeve positioning tube 16 (collectively a retrieval catheter) and (ii) filters 24, 26 mounted at the distal end of a filter guidewire 22. The retrieval system 10 is shown in U.S. application Ser. No. 12/738,702, which is herein incorporated by reference. The capture sleeve 14 is radially expandable within the blood vessel proximal to the clot. Likewise, the filters 24, 26 are radially expandable within the blood vessel distal to the clot before being pulled through the clot to capture or entrain the clot material. The filters 24, 26 and captured clot are then pulled into the capture sleeve 14, and the capture sleeve 14 encloses the filters 24, 26 and the captured clot material. The capture sleeve 14, filters 24, 26, and the captured clot material are then pulled into a capture/delivery sheath 12 for removal from the body.

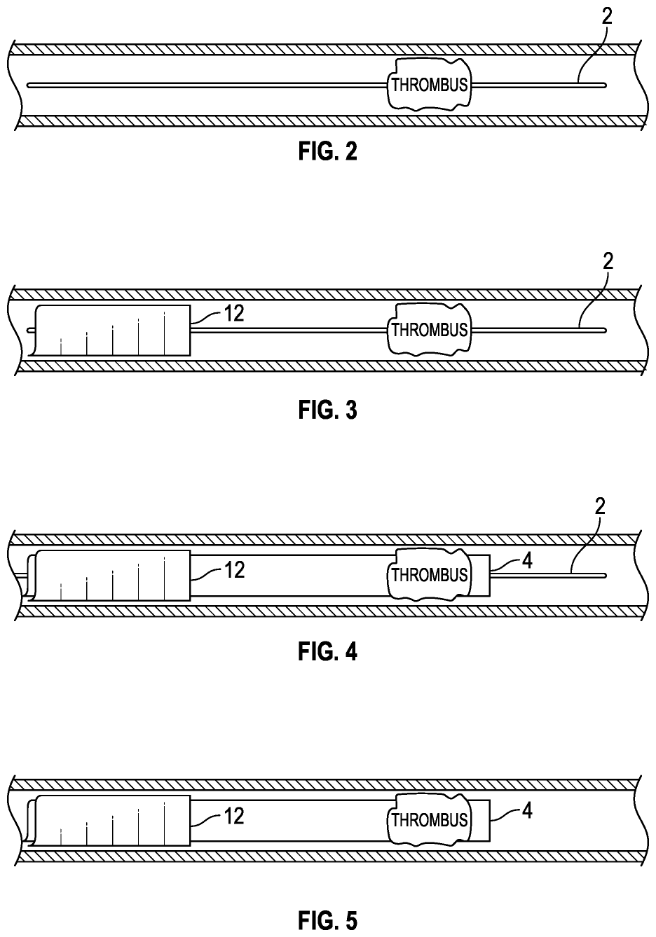

As shown in FIG. 2, the retrieval system 10 is deployed at the clot by initially locating a positioning guidewire 2 and the capture/delivery sheath 12 at the treatment site. The positioning guidewire 2 is located within the blood vessel by navigating the distal end of the positioning guidewire 2 from an access site to the clot. The positioning guidewire 2 is pushed through or around the clot to position the distal end of the positioning guidewire 2 distal to the clot. As shown in FIG. 3, the capture/delivery sheath 12 is guided to the clot over the positioning guidewire 2 to position the distal end of the capture/delivery sheath 12 proximate to the clot. After initial positioning of the capture/delivery sheath 12, the capture/delivery sheath 12 remains in place throughout the procedure including when the filter 24 is redeployed for multiple passes through the clot. However, as discussed further below, the positioning guidewire 2 is removed from the body each time the filters 24, 26, coupled along a filter guidewire 22, are deployed within the blood vessel.

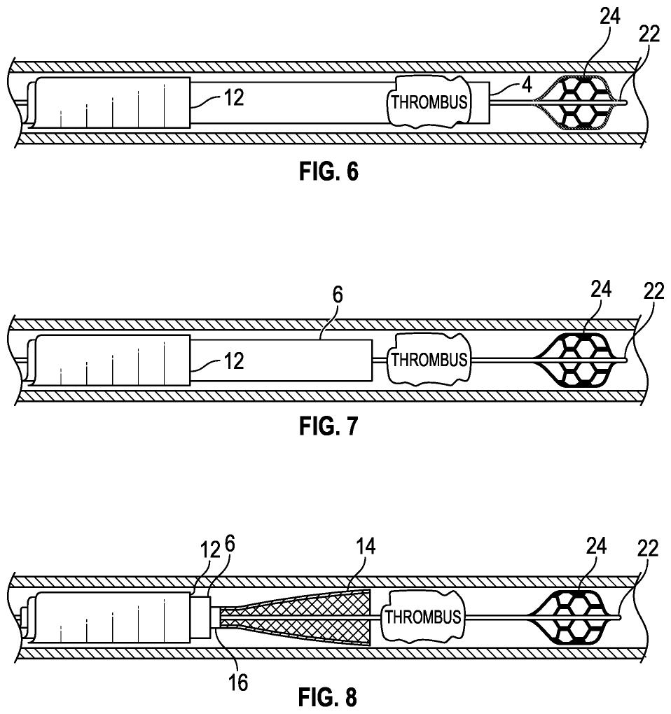

As shown in FIG. 4, the filters 24, 26 are deployed by navigating a filter delivery catheter 4 through the catheter/delivery sheath 12 and over the positioning guidewire 2 to the clot. The distal end of the filter delivery catheter 4 exits the catheter/delivery sheath 12 before continuing over the positioning guidewire 2 across or around the clot. The positioning guidewire 2 serves as a guide for navigating the filter delivery catheter 4 to and past the clot. As shown in FIG. 5, after the filter delivery catheter 4 is positioned, the positioning guidewire 2 is withdrawn from the filter delivery catheter 4 and the body to permit the insertion of a filter guidewire 22 (having the filters 24, 26) through the filter delivery catheter 4. The filter guidewire 22 is then navigated to the clot. As shown in FIG. 6, the filter guidewire 22 is passed through the filter delivery catheter 4 until the filter 24 exits the filter delivery catheter 4 distal to the clot. The filter delivery catheter 4 is sized such that the filter 24 is constrained and collapsed while the filter 24 is moved through the filter delivery catheter 4. The filter 24 self-expands upon exit from the filter delivery catheter 4 once freed from the filter delivery catheter 4. The filter delivery catheter 4 is then withdrawn from the body through the capture/delivery sheath 12.

As shown in FIG. 8, the flexible capture sleeve 14 is deployed proximal to the clot by navigating the capture sleeve 14 on the distal end of the capture sleeve positioning tube 16 to the clot, for instance while within the capture sleeve sheath 6 (see FIG. 7). The flexible capture sleeve 14 is delivered constrained within the distal end of the capture sleeve sheath 6. The capture sleeve sheath 6 is sized to constrain and collapse the flexible capture sleeve 14 when the flexible capture sleeve 14 is held within the capture sleeve sheath 6. The capture sleeve sheath 6 is navigated through the capture delivery sheath 12 until the distal end of the capture sleeve sheath 6 exits the capture/delivery sheath 12 and is positioned proximate to the clot (see FIG. 7). As shown in FIG. 8, the capture sleeve 6 is then slid axially in the proximal direction to uncover the capture sleeve 14 permitting the capture sleeve 14 to self-expand. The filter 24 is then pulled proximally through the clot to engage the clot material and draw the captured clot material into the capture sleeve 14 for removal from the body through the capture/delivery sheath 12.

As discussed above, certain clots may require multiple deployments of filters 24 (or filters 24, 26) distal to the clot and multiple passes of the filters 24, 26 through the clot to fully extract the clot or remove sufficient clot material to restore blood flow through the vessel. After each pass through the clot, the filters 24, 26 and the capture sleeve 14 are removed from the body for cleaning. Each pass of the filters 24, 26 includes a re-deployment of the filters 24, 26 and the capture sleeve 14 on opposing sides of the clot. The filters 24, 26 are collapsed, positioned within the filter delivery catheter 4, and the filter guidewire 22 with collapsed filters 24, 26 are re-delivered to the clot, for instance through the filter delivery catheter 4. To deliver the collapsed filters 24, 26 distal to the clot, the positioning guidewire 2 is first re-inserted and navigated distally past the clot before the filter delivery catheter 4 is navigated past the clot and over the previously navigated positioning guidewire 2.

Re-insertion (and the preceding initial insertion) are conducted carefully to avoid injury to the blood vessel and to consistently position the distal end of the positioning guidewire 2 at a specified location within the blood vessel. However, the clot itself may cause the positioning guidewire 2 to deflect during insertion and each re-insertion as the positioning guidewire 2 engages with the clot while attempting to cross the clot. Accordingly, the clot may frustrate the accurate positioning and consistent repositioning of the positioning guidewire 2 in one or more of the initial insertion or re-insertions. The insertion and reinsertion procedures and attendant positioning of the positioning guidewire 2 (as well as the separate filter delivery guidewire 22 having the filters 24, 26) in each of these procedures add complexity and potentially risky steps to the thrombectomy procedure that are repeated for each pass of the filter 24 (or filters 24, 26) through the clot. As noted herein, each pass of the filter 24 includes withdrawal of the filter 24 and the filter guidewire 22 from the previous pass, re-insertion of the positioning guidewire 22, and re-insertion of the filter guidewire 22 and filter 24 (cleaned). Accordingly, the repeated insertion, re-insertion increases procedure time, is laborious and increases the potential for patient complications.

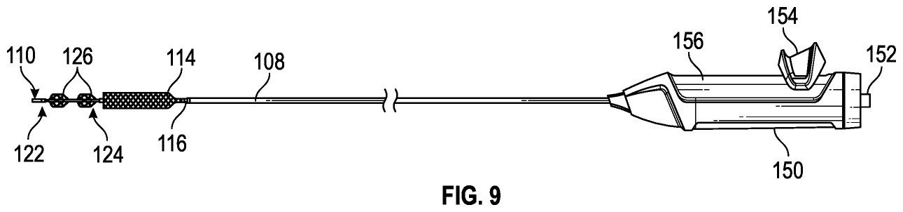

An over-the-wire retrieval system 110 is shown in FIG. 9. The over-the-wire retrieval system 110 includes (i) a flexible capture sleeve 114 coupled with a distal portion of a flexible sleeve positioning tube 116 and (ii) a filter assembly 124 coupled with a filter catheter 122 in contrast to the filter guidewire 22 previously noted above. The filter catheter 122 includes a lumen for slidably receiving a positioning guidewire 102 to permit navigation of the filter catheter 122 along the positioning guidewire 102. Likewise, the flexible sleeve positioning tube 116 defines a lumen for slidably receiving the filter catheter 122 and permits the delivery of the flexible sleeve positioning tube 116 over the filter catheter 122. In this configuration, the filter catheter 122 and the flexible sleeve positioning tube 116 are each delivered and retrieved over the positioning guidewire 102 without removing or repositioning the positioning guidewire 102 from the blood vessel, for instance to permit the delivery of a filter guidewire 22 in the previous example above.

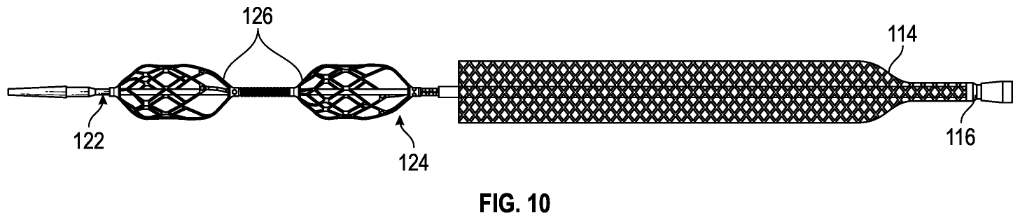

As illustrated in FIG. 10, the capture sleeve 114 in an example includes one or more of a woven material, fenestrated structure, or other filtering structure configured to radially expand (including self-expansion) when unconstrained. In one example, the capture sleeve 114 self-expands within the blood vessel proximate to the clot to fill the vessel or otherwise obstruct the vessel proximal to the clot to prevent the passage of clot while permitting blood flow. The term “clot” used herein and with respect to embodiments of the present invention refers to any clot material within a blood vessel including, but not limited to, one or more of thrombus or emboli; and clot, thrombus and emboli are used interchangeably. Likewise, the filter baskets 126 include a fenestrated structure or other filtering structure configured to radially expand (including self-expansion) when unconstrained to fill or otherwise obstruct the vessel proximal to the clot while permitting blood flow. The term “filter” used here and with respect to embodiments of the present invention refers to woven, braided, fenestrated, or permeable structure capable of mechanically engaging and capturing clot while permitting the flow of blood through the filter baskets 126. As shown in FIG. 9, the filter catheter 122 may comprise multiple filter baskets 126 to enhance clot capture and minimize the loss of clot fragments with each pass. In other embodiments, the filter catheter 122 may comprise a single filter basket 126 to account for complex or tortuous geometry, such as in pulmonary arteries where pulmonary embolisms may become lodged. After deployment of the one or more filter baskets 126, the filter baskets 126 are pulled through the clot to engage the clot material. The one or more filter baskets 126 (with captured clot material) are moved into the capture sleeve 114 to trap the filter baskets 126 and the clot material within the capture sleeve 114. The capture sleeve 114, the one or more filter baskets 126, and the captured clot are then, in one example, pulled into the capture/delivery sheath 112 (see FIG. 18) for removal from the body.

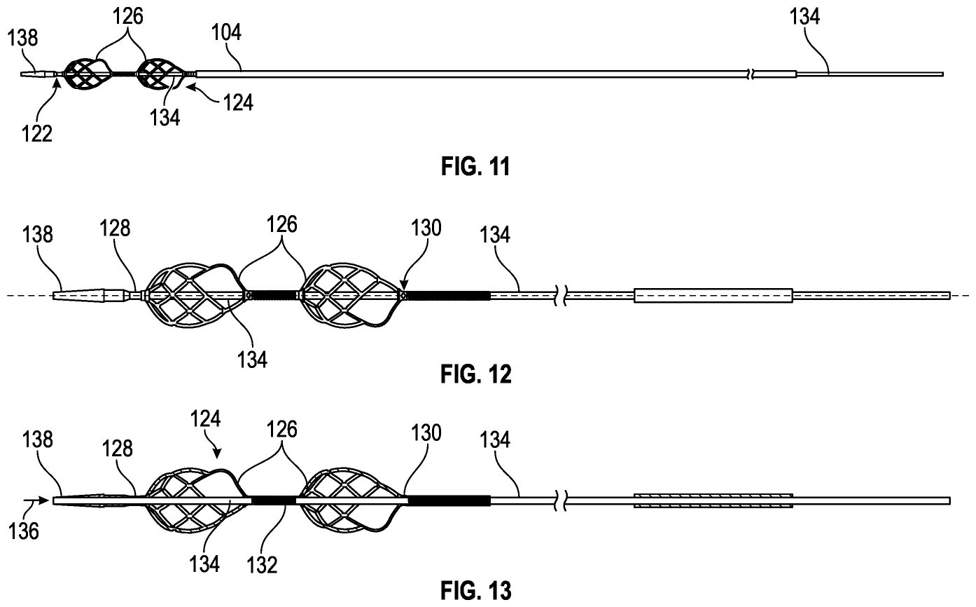

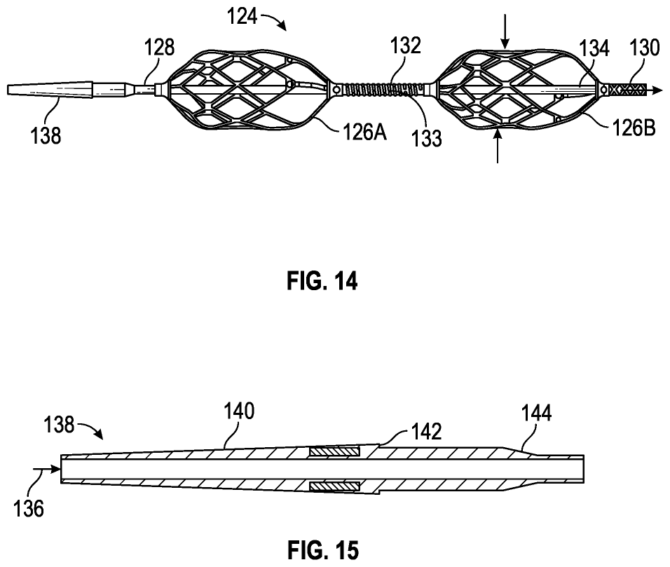

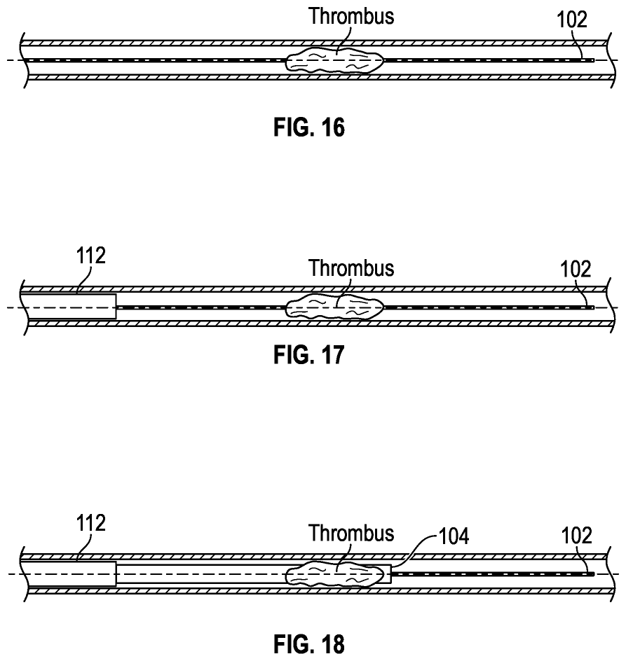

As shown in FIGS. 11-13, the filter catheter 122 includes a catheter shaft 134 defining a central lumen 136 (see FIG. 13). A filter assembly 124 and an atraumatic tip 138 are positioned at the distal end of the catheter shaft 134 (see FIG. 14). As illustrated in FIGS. 11 and 12, a filter delivery catheter 104 can define a central lumen for receiving the catheter shaft 134, which permits translation of the filter delivery catheter 104 axially along the central shaft 130 of the filter catheter 122. The central lumen 136 of the filter catheter 122 is sized to receive a positioning guidewire 102 (See FIG. 16 and later) such that the filter catheter 122 is slidable over the positioning guidewire 102 to the treatment site. The filter catheter 122 is navigated to the treatment site over the positioning guidewire 102, used captures clot, is removed from the body and redeployed without removing or repositioning of the positioning guidewire 102. The positioning guidewire 102 in various examples is between around 50 and 300 cm in length and has a diameter of around 0.014″, 0.018″, or 0.035″ or similar. The positioning guidewire 102 acts as a rail or guide for other components of the retrieval system 110 to translate there along toward the treatment site. The positioning guidewire 102 is slidably received within the central lumen of the catheter shaft 134 of the filter catheter 122. The filter catheter 122 and its catheter shaft 134 optionally serves as a rail or guide for other components of the retrieval system 110. In at least one example, the interior surface, exterior surface, or both surfaces of the catheter shaft 134 of the filter catheter 122 is coated with a lubricious coating to ease sliding movement of the components over the catheter shaft 134.

As shown in FIGS. 13 and 14 the filter assembly 124 includes at least one filter basket 126 having a fixed collar 128 and a sliding collar 130. The fixed collar 128 is, in one example, statically coupled to the catheter shaft 134. The sliding collar 130 is movably coupled to the catheter shaft 134 to permit translational movement of the sliding collar 130 and translational movement of a portion of the filter assembly 124 (e.g., between the collars 128, 130). In this configuration, the sliding collar 130 is translatable axially away from the fixed collar 128 when the filter basket 126 is compressed radially. The translation of the sliding collar 130 thereby permits the one or more filter baskets 126 to collapse radially.

In another example, the filter basket 126 having one or more component baskets is constructed with a shape memory metal, fenestrated structure, or other elastic material that biases the filter basket 126 toward the expanded position (shown FIGS. 13 and 14) while the filter basket 126 is unconstrained, for instance by the capture sleeve sheath 108 (See FIG. 9). When unconstrained (e.g., with the capture sleeve sheath 108 withdrawn) the biased filter basket 126 expands and the sliding collar 130 translates axially toward the fixed collar 128 to permit the radial expansion. In at least one example, at least one of the fixed collar 128 or the sliding collar 130 is notched or etched to form a fenestrated or corrugated structure (e.g., with ridges, knurling, corrugations or the like). The fenestrated structure is, in one example, shaped to increase friction (e.g., by point loading the engagement of the fenestrated or corrugated with the catheter shaft 134) and enhance static coupling between the fixed collar 128 and the catheter shaft 130. Conversely, the fenestrated structure is, in another example, shaped to reduce the contact area between the sliding collar 130 and the catheter shaft 134 to minimize friction between the collar 130 and the shaft 134 and promote translating relative movement.

As depicted in FIGS. 13 and 14, in at least one example, the filter assembly 124 can comprise at least two component filter baskets 126A, 126B connected by an intermediate collar 132. In at least one embodiment, the filter baskets 126A, 126B and intermediate collar 132 include a unitary fenestrated body, such as a laser cut hypotube. The intermediate collar 132 is, in one example, movably coupled along the catheter shaft 134. As shown in FIG. 14 one filter basket (filter 126A as depicted in FIG. 14) includes the fixed collar 128 statically coupled to the catheter shaft 134 and a movable intermediate collar 132 interconnecting the filter basket 126A to the other filter basket 126B. The other basket 126B is movably connected to the catheter shaft 134 with the intermediate collar 132 and a slidable collar 130. The fixed collar 128 of filter basket 126A positions both filter baskets 126A, 126B for radial expansion and compression proximate a distal end portion of the catheter shaft 134 while the slidable intermediate collar 132 and the slidable collar 130 permits movement of both filter baskets 126A, 126B to radially compress and expand.

In another example, the static coupling provided with the fixed collar 128 braces the one or more filter baskets 126A, B during translations of the baskets 126A, B, for instance into the capture sleeve 114. As the catheter shaft 134 of the filter assembly 124 is drawn proximally, the filter baskets 126A, B are correspondingly drawn proximally. The filter baskets 126A, B engage with clot material and continued proximal movement of the catheter shaft 134 pulls the baskets and clot material into the capture sleeve 114. The movable couplings provided with intermediate collar 132 and the slidable collar 130 permits axial compression of the filter assembly 124 and its baskets 126A, B toward the static fixed collar 128. The baskets 126A, B accordingly compress with proximal movement of the catheter shaft, the associated elements of the baskets (e.g., wires, filaments, mesh or the like) aggregate over each other, and fenestration openings effectively shrink. The capture of the clot material is accordingly enhanced with the aggregated elements of the baskets 126A, B. Further, with additional proximal movement of the catheter shaft the filter assembly 124 compresses until gaps in the intermediate collar (described herein below), and fenestration openings in the baskets 126A, B or the like, are compressed and the material of the baskets 126A, B and the intermediate collar 132 stacks (e.g., a compression limit). The fixed collar 128 braces the baskets 126A, B and the intermediate collar 132 against further movement and instead transmits the proximal movement of the catheter shaft 134 fully to the baskets and drives the baskets into the capture sleeve 114.

As illustrated in FIG. 13, the fixed (distal) collar 128 is fixed to the catheter shaft 134 while the slidable (proximal) collar 130 and the intermediate collar 132 are movable along the catheter shaft 134. In various embodiments, different combinations of collars 128, 130, 132 are movable or static relative to the catheter shaft 134. For instance, in one example, the distal collar 128 and intermediate collar 132 are movable over the catheter shaft 134, and the proximal collar 130 is statically coupled with the catheter shaft 134. In another example, the intermediate collar 132 is statically coupled on the catheter shaft 134 while the distal collar 128 and the proximal collar 130 are movably coupled along the catheter shaft 134.

In at least one example, the intermediate collar 132 includes notches 133 (also referred to as gaps, openings or the like) that permit axial expansion or compression of the intermediate collar 132. The permitted expansion or compression provides a deflectable quality (e.g., pliability, compressibility or the like) to intermediate collar 132 and permits it to behave similar to a spring between filter baskets 126A, 126B. The intermediate collar 132 also allows filter baskets 126A, 126B to move relative to each other to absorb and minimize stress that may cause damage to the filter assembly 124 as the filter assembly 124 is pulled through the clot. Additionally, the relative movement permits the application of a consistent force on the catheter shaft 134 while minimizing one or both of sudden engagement with thrombus and seizing of the catheter and sudden disengagement of the filter baskets 126A, B from thrombus. Instead, the intermediate collar 132 provides compression (as well as expansion) that permits the gradual escalation of force from the catheter shaft 134 to the filter baskets 126A, B and the associated thrombus during engagement and after engagement (e.g., when the thrombus is pulled). As noted above, the intermediate collar 132 and the filter baskets 126A, B optionally axially compress during capture of clot material and proximal movement in a manner that aggregates elements of the filter baskets 126A, B and enhances capture and driving of the clot material into the capture sleeve 114. In another example, the filter baskets 126A, 126B simultaneously engage different portions of the clot material as the filter baskets 126A, 126B are pulled through the clot and engage clot material. For example, one filter basket 126A (or a portion of the basket 126A) engages more organized and difficult to move thrombus material while filter basket 126B (or a portion thereof) engages less organized and easier to move thrombus material. The flexibility provided with the intermediate collar 132 (e.g., as a biasing element, shock absorber, damping element or the like), as well as the deformability of the baskets 126A, B themselves, modulates the application of force to each type of thrombus in a manner that permits retrieval of thrombus having thrombus material with a range of consistencies without sudden engagement and disengagement.

As illustrated in FIG. 15, the atraumatic tip 138 can comprise a front sloped portion 140. The front sloped portion 140 can be angled to ease insertion of the filter catheter 122 into the capture/delivery sheath 112 and to decrease engagement or snagging of these structures, for instance as the filter catheter 122 is loaded into the capture/delivery sheath 112. The shape of the atraumatic tip 138 eases navigation of the filter catheter 122 across the thrombus. In at least one embodiment, the outer diameter of the front sloped portion 140 (e.g., a taper) corresponds to the outer diameter of the filter delivery catheter 104 (See FIG. 11). The filter delivery catheter 104 is slid axially over the filter catheter 122 until the sloped portion 140 protrudes from the distal end of the filter delivery catheter 104 and the remainder of the filter catheter 122 is concealed within the catheter 104. In this configuration, the atraumatic tip 138 operates as a dilator for the filter delivery catheter 140 and facilitates navigation and delivery of the filter delivery catheter 104 and the filter catheter 122 to the treatment site and across the thrombus together. As further shown in FIG. 15, the atraumatic tip 138 optionally includes a shoulder 142 for engaging the distal end of the filter delivery catheter 104, and in one example provides a flush (including near flush) interface between the catheters 104, 122. In another example, the atraumatic tip 138 includes a rear sloped portion 144 (e.g., a taper) for aligning and guiding the distal end of the filter delivery catheter 104 into engagement with the shoulder 142.

As illustrated in FIG. 9, in at least one embodiment, the over-the-wire retrieval system (OTW retrieval system) 110 includes a handle 150 for operating the OTW retrieval system 110. The proximal end of the capture sleeve positioning tube 116 is statically coupled to the proximal end of the handle 150 and extends through the handle 150 to provide a central lumen through the handle 150. In this configuration, the handle 150 includes a seal or Luer connector 152 for controlling access to the proximal end of the capture sleeve positioning tube 116. The capture sleeve positioning tube 116 is coaxially positioned within the capture sleeve sheath 108 wherein the proximal end of the capture sleeve sheath 108 is received within the handle 150. The handle 150 shown in FIG. 9 includes an operator, such as a slider 154, attached to the proximal end of the capture sleeve sheath 108. The slider 154 is moveable (e.g., axially in this example) to move the capture sleeve sheath 108 axially relative to the capture sleeve positioning tube 116 (the sheath 108, position tube 116 and capture sleeve 114 are collectively a retrieval catheter). In at least one example, the handle 150 includes a track 156 coupled with the slider 154, and the track 156 guides movement of the slider 154. Optionally, the handle 150 includes a locking mechanism that biases the slider 154 toward a secure position that correspondingly secures the associated capture sleeve sheath 108 in position. Accordingly, the operator moves the capture sleeve sheath 108 with operation of the slider 154, and upon release of the slider 154 the capture sleeve sheath 108 remains in a static position (e.g., relative to the capture sleeve positioning tube 116). FIGS. 16-24 illustrate operation of the OTW retrieval system 110 for retrieval of thrombus (e.g., a clot). As shown in FIG. 16, a positioning guidewire 102 is navigated through a blood vessel from an access site toward a treatment site, for instance having the thrombus. The positing guidewire 102 is pushed across or around the thrombus to position the distal end of the positioning guidewire 102 distal relative to the thrombus. The positioning guidewire 2 operates as a rail or guide for navigating the various components of the OTW retrieval system 110 proximate to the thrombus (e.g., proximal, distal and through or around the thrombus). The positioning guidewire 102 is, in one example, conventionally dimensioned, for instance with a diameter of between around 0.018″ to 0.035″ and a length between around 50 to 260 cm. In certain embodiments, the positioning guidewire 102 includes a hydrophilic coating for easing navigation of the positioning guidewire 102 through the blood vessel and to facilitate the movement of components of the retrieval system 110 over the positioning guidewire 102.

As depicted in FIG. 16, the positioning guidewire 102 is navigated through the thrombus. In another example, the positioning guidewire is navigated through the thrombus or between the thrombus and the vessel wall, for instance depending on the density of the thrombus. The distal end of the positioning guidewire 102 extends a specified distance past the thrombus to provide a runway for deployment of various components of the OTW retrieval system 110 distal to the thrombus. Additionally, the positioning of the distal end of the positioning guidewire 102 secures the positioning guidewire 102 in place and minimizes inadvertent withdrawal of the guidewire 102 through the thrombus (e.g., proximally). Unlike the other retrieval system 10 discussed herein having the guidewire 2 that is removed and re-inserted in each pass, the positioning guidewire 102 for the current OTW retrieval system 110 remains in place for the procedure including potential multiple passes of the filter delivery catheter 104.

As shown in FIG. 17, the capture/delivery sheath 112 is guided through the vessel to the thrombus over the positioning guidewire 102. The distal end of the capture/delivery sheath 112 is positioned proximal to the thrombus (e.g., a specified distances to permit deployment of one or more other features of the system 110). Once positioned, the components of the retrieval system 110 are navigated through the vessel and within the lumen of the capture/delivery sheath 112 to the thrombus over the positioning guidewire 102. As shown in FIG. 17, the distal end of the capture/delivery sheath 112 is proximally spaced from the thrombus to permit deployment of various components of the OTW retrieval system 110 between the thrombus and the distal end of the capture/delivery sheath 112. After the capture/delivery sheath 112 is positioned, the capture/delivery sheath 112 optionally remains in place with the positioning guidewire 102 throughout the procedure for navigating and retrieving the various components of the OTW retrieval system 110 to and from the treatment site within the vessel. Accordingly, the positioning guidewire 102 and the capture/delivery sheath 112 are configured to provide inner and outer guidance of components to the treatment site; inner with components coupled over the positioning guidewire 102 and outer with the capture/delivery sheath 112 surrounding the components.

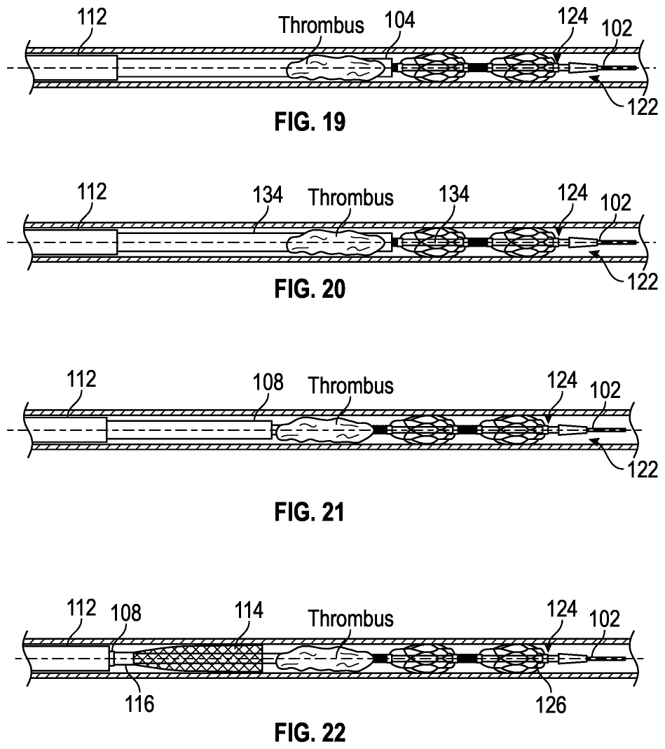

As shown in FIG. 18, the filter delivery catheter 104 is guided over the positioning guidewire 102 and through the catheter/delivery sheath 112. The filter delivery catheter 104 exits the catheter/delivery sheath 112 proximate to the thrombus in FIG. 19. The filter delivery catheter 104 is further advanced axially over the positioning guidewire 102. As shown in FIG. 19 the distal end of the filter delivery catheter 104 is positioned distal to the thrombus. In some examples thrombus may be more organized, dense or the like and accordingly more difficult to cross. The positioning guidewire 102 maintains the general alignment of the filter delivery catheter 104 with the vessel walls and minimizes the deflection of the filter delivery catheter 104 as the filter delivery catheter 104 is pushed across the thrombus. For example, the positioning guidewire 102 provides guidance to the delivery catheter 104 to ensure delivery through the vessel in contrast to engagement with the vessel wall.

As shown in FIG. 19, the filter catheter 122 includes a central lumen for receiving the positioning guidewire 102. The filter catheter 122 is navigated over the positioning guidewire 102 and through the filter delivery catheter 104 to the treatment site, for instance distal to the thrombus as shown. The filter catheter 122 includes a filter assembly 124 positioned proximate to a distal end of the filter catheter 122. The filter catheter 122 is navigated through the filter delivery catheter 104 until the filter assembly 124 exits the filter delivery catheter 104. The filter assembly 124 comprises at least one filter basket 126 (two component filter baskets are shown in FIG. 19) configured to expand (including self-expand) when unconstrained by the filter delivery catheter 104. As shown in FIG. 19 the example filter baskets 126 self-expand upon exiting the filter delivery catheter 104.

As shown in FIGS. 19 and 20, the filter delivery catheter 104 is in one example pulled proximally across the thrombus to uncover the filter baskets 126 and the catheter shaft 134 of the filter catheter 122. The filter baskets 126 are accordingly deployed distal to the thrombus to facilitate capture and retraction of the thrombus into the capture/delivery sheath 112. As discussed herein, the filter baskets 126 are withdrawn into the capture/delivery sheath 112 when withdrawal of the system 110 is specified. In at least one embodiment, the filter delivery catheter 104 is fully withdrawn from the body through the capture/delivery sheath 112, for instance to provide space within the central lumen of the capture/delivery sheath 112 for the delivery of various other components of the OTW retrieval system 110 and for the withdrawal of thrombus. In at least one embodiment, the filter delivery catheter 104 is retracted proximally to deploy the filter baskets 126 but is not otherwise retracted from the body. In this configuration, the filter delivery catheter 104 is optionally moved forward (relative to the baskets 126) in the distal direction to constrain and collapse the filter baskets 126, for instance for repositioning of the baskets 126. In another example, the filter baskets 126 are moved proximally to constrain and collapse the filter baskets 126.

As shown in FIG. 21, after the filter delivery catheter 104 is optionally withdrawn a capture sleeve sheath 108 is navigated over the shaft of the filter delivery catheter 104 and through the capture/delivery sheath 112 to the treatment site. The distal end of the capture sleeve sheath 108 is navigated distally until the distal end of the capture sleeve sheath 108 exits the capture/delivery sheath 112 and is positioned proximate to the thrombus. As illustrated in FIG. 21, a distal end portion of the capture sleeve sheath 108 is positioned adjacent to the proximal side of the thrombus. A capture sleeve 114 is attached to the distal end of a capture sleeve positioning tube 116 nested within the capture sleeve sheath 108. The capture sleeve positioning tube 116 is navigated over the shaft of the filter delivery catheter 104 and through the capture sleeve sheath 108 to the treatment site. In certain embodiments, the capture sleeve positioning tube 116 and the capture sheath 108 are navigated to the treatment site. In this configuration, during navigation, the capture sleeve 114 is positioned within the distal end of the capture sheath 108 such that the capture sheath 108 maintains the capture sleeve 114 in a collapsed configuration during navigation of both devices. The capture sleeve positioning tube 116, capture sleeve 114 and the capture sleeve 108 are in some examples referred to as a retrieval catheter.

As shown in FIG. 22, the capture sleeve sheath 108 is pulled in the proximal direction to expose the capture sleeve 114 and deploy the capture sleeve 114. The capture sleeve 114 is, in one example, configured to self-expand when exposed by the proximal movement of the capture sleeve sheath 108, for instance with shape memory materials, return bias provided by the previously constrained capture sleeve 114 or the like. The capture sleeve 114 is configured to at least partially fill the vessel while permitting blood flow and receiving thrombus engaged by the at least one filter 126 and driven toward the sleeve 114. The filter assembly 124 including the filters 126 is pulled proximally to engage at least a portion of the thrombus with the one or more filters 126 and pull the thrombus into the deployed capture sleeve 114. Once the thrombus is pulled into the capture sleeve 114, the capture sleeve 114 (with captured thrombus and the one or more filters 126 therein) are pulled into the capture/delivery sheath 112 and withdrawn from the body.

In certain situations, the filter 126 can miss the thrombus or pulls a small portion of the overall thrombus into the one or more filters 126. In this arrangement, the filter delivery catheter 104 (See FIGS. 18 and 19) is optionally moved distally over the one or more filters 126 to “re-sheath” the filters 126 including collapse of the filters 126 with the filter delivery catheter 104 after the filters 126 are within the capture sleeve 114. Thrombus material already entrained in the one or more filters 126 is macerated or wrung from the filters 126 as the filters 126 are collapsed by the filter delivery catheter 104. The filter delivery catheter 104 and the re-sheathed filter 126 are navigated over the positioning guidewire 102, past the thrombus, for another pass to capture additional thrombus. Re-sheathing and re-deployment and capture of additional thrombus are conducted without fully removing and re-deploying one or more components of the OTW retrieval system 110, for instance, the positioning guidewire 102, the filter delivery catheter 124 or the like. Instead, these components remain proximate to the thrombus, and are reset (e.g., re-sheathed) for immediate use to capture and withdraw additional thrombus.

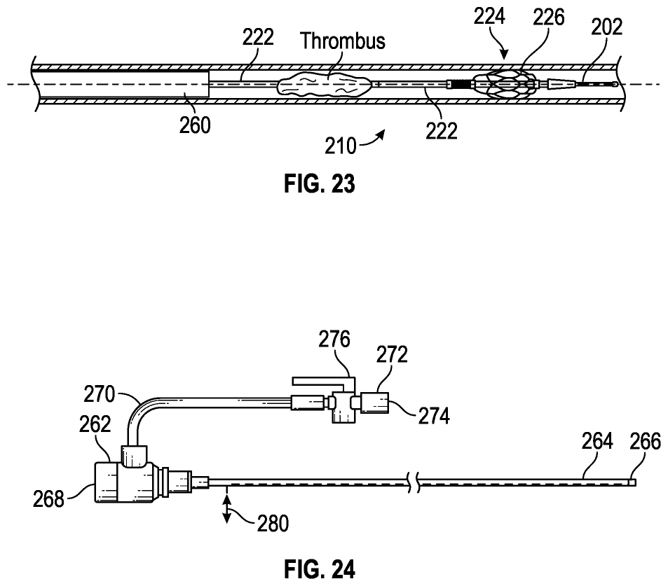

Another example OTW retrieval system 210 is shown in FIG. 23. The OTW retrieval system 210 includes a filter assembly 224 mounted on a filter catheter 222 and (ii) an aspiration sheath 260. In this configuration, the filter assembly 224 includes at least one filter 226. As shown, the at least one filter 226 is deployed distal to the illustrated thrombus, for instance with retraction of a filter delivery catheter (e.g., similar to the filter delivery catheter 104 shown in FIG. 19). The at least one filter 226 is configured to expand within a vessel distal to the thrombus to fill the vessel and prevent passage of clot material past the filter 226. The at least one filter 226 is fenestrated and permits blood flow therethrough. The expanded filter assembly 224 is pulled toward the thrombus to engage thrombus and draw the thrombus toward the mouth of the aspiration sheath 260. A negative pressure (e.g., vacuum) is supplied by the aspiration sheath 260 at its open distal end to aspirate the clot material into the aspiration sheath 260. Accordingly, the aspiration sheath 260 ‘cleans’ thrombus from the at least one filter 226 and, as noted herein, prepares the at least one filter 226 for re-deployment and capture of additional thrombus. In one example, the positioning of the thrombus proximate to the opening of the aspiration sheath 260 permits the application of a lesser negative pressure because of the thrombus proximity. This minimizes the aspiration of blood, body fluids or the like and minimizes vacuum engagement of the aspiration sheath 260 with the vessel wall. In contrast, other aspiration systems apply greater negative pressure to aspirate thrombus material remote to an aspiration catheter opening and unintentionally withdrawn additional blood and body fluids.

As shown in FIG. 23, the filter catheter 222 includes one or more filter baskets 226 having a fenestrated structure or other structure that permits blood flow while capture thrombus. The filter catheter 222 biases the one or more filter baskets 226 to radially deploy when unconstrained. For instance, the one or more filter baskets 226 are configured to self-expand from a constrained configuration during delivery to a deployed configuration when unconstrained (e.g., with retraction of a filter delivery catheter). As illustrated in FIG. 23, in at least one embodiment, the filter catheter 222 includes at least one filter basket 226. To accommodate the geometry of pulmonary vessels, the filter basket 226 includes a complementary geometry, including, but not limited to, a spherical geometry, cylindrical geometry or the like as depicted in FIG. 23 when expanded. The one or more filter baskets 226 are deformable and conform to variations in pulmonary vessel walls to fit within and consistently engage along vessel walls. In certain embodiments, the filter catheter 222 comprises a plurality of filter baskets 226 arranged axially along the filter catheter 222.

The filter catheter 222, similar to the filter catheter 122, includes a lumen for movably receiving a positioning guidewire 202 for delivery and retrieval of the filter catheter 222 over the positioning guidewire 202. The filter catheter 222 is navigated over the positioning guidewire 202 to the thrombus and across the thrombus to position the filter assembly 224 distal to the thrombus. The filter catheter 222 is optionally conveyed within a filter delivery catheter (e.g., see filter delivery catheter 104 in FIGS. 18 and 19). The filter delivery catheter constrains the at least one filter basket 226 of the filter assembly 224 in a collapsed configuration for delivery. Once the collapsed filter basket 226 is positioned distal to the thrombus, the filter delivery catheter is retracted to uncover the filter basket 226, and the filter basket 226 expands radially distal to the thrombus. In certain embodiments, the one or more filter baskets 226 are configured for re-sheathing in situ. For instance, the filter delivery catheter 204 is moved over the deployed one or more filter baskets 226 to constrain and collapse the filter baskets 226. The re-sheathed and collapsed one or more filter baskets 226 are moved distal to the thrombus (e.g., over the positioning guidewire 202) for redeployment of the filter baskets 226 distal to the clot for a supplemental pass (or passes) through the thrombus. The re-sheathing permits resetting of the one or more filter baskets 226 for rapid redeployment proximate to the thrombus. For example, re-sheathing permits rapid multiples passes of the filter basket 226 through the thrombus and over the positioning guidewire 202. In operation, the one or more filter baskets 226 quickly “feeds” thrombus material to the opening of the aspiration sheath 260 for aspiration and removal from the blood vessel. The suction applied through the aspiration sheath 260 and available at the opening of the sheath permits the aspiration sheath 260 to continually extract thrombus as the thrombus material is brought to the opening of the aspiration sheath 260 by the one or more filter baskets 226. The aspiration sheath 260 ‘cleans’ the one or more filter baskets 226, and optionally the OTW retrieval system 210 is operated to re-sheath the filter baskets 226 for redeployment proximate to the thrombus, capture of additional thrombus and repeated aspiration or ‘cleaning’ of the thrombus from the filter baskets 226.

As shown in FIG. 24, the aspiration sheath 260 includes, in an example, a main hub 262 and a sheath body 264 that extends distally from the main hub 264. The sheath body 264 includes a lumen 266 accessible through the main hub 262. The sheath body 264 optionally includes a secondary lumen for the delivery of diagnostic or therapeutic agents to the treatment site separate from the lumen 266. In one example, the lumen 266 is configured for receipt of one or more components of the OTW retrieval systems discussed herein, and for capture and removal of thrombus. In at least one example, the interior surfaces, exterior surfaces, or both surfaces the sheath body 264 are coated with a lubricious coating to ease sliding movement of components through the sheath body 264 and through vasculature. In at least one embodiment, the main hub 264 includes a hemostasis valve 268 to seal the proximal end of the lumen 266 of the sheath body 262. In an embodiment, the aspiration sheath 260 includes a side path 270 extending from a secondary hub 272 to the main hub 264. The side path 270 includes side lumen 274 accessible through the secondary hub 272. The distal end of the side lumen 274 of the side path 270 is fluidly connected to the lumen 266 of the sheath body 264. In at least one embodiment, a syringe is connected to the secondary hub 272 to apply a negative pressure through the side lumen 274 of the side path 270 and the lumen 266 of the sheath body 264. The applied negative pressure draws thrombus material into the distal opening of the sheath body 264 as discussed herein. The side path 270 optionally includes a manual valve 276 that selectively controls the application of negative pressure through the side lumen 274 of the side path 270 and correspondingly the lumen 266 of the sheath body 264.

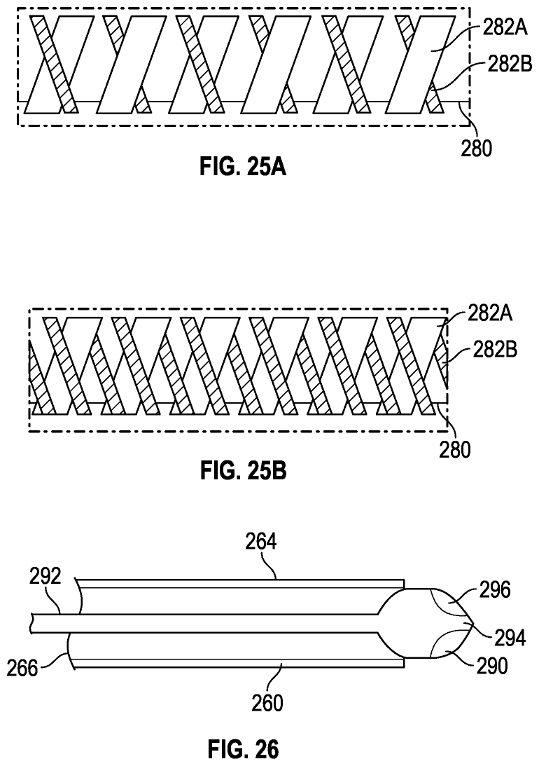

As shown in FIGS. 25A-B, the sheath body 264 of the aspiration sheath 260 optionally includes a braided structural layer to provide flexibility and pushability for the sheath body 264. As illustrated in FIGS. 25A-B, the braided structural layer includes at least a first filar array 282A and a second filar array 282B. The first filar array 282A is interwoven into the braided layer such that the filars of the first filar array 282A are arranged in a first direction while the second filar array 282B is interwoven into the braided layer such that the filars of the second filar array 282B are arranged in a second direction transverse to the first direction (e.g., at an angle to the first direction). In an example, the first filar array 282A includes at least one first braid wire have a cross-section of between around 0.003″×0.007″ to around 0.005″×0.015″. The second filar array 282B includes at least one second braid wire having a cross-section of between around 0.001″×0.003″ to around 0.003″×0.008″. In an example, the first filar array 282A and the second filar array 282B interwoven in an alternating 1:1 pattern as shown FIG. 25A. In another example, the first filar array 268A and the second filar array 282B are interwoven in an unbalanced fashion as shown FIG. 25B, for instance having greater number of filars in the second filar array 282B or a higher density (e.g., pics per inch) of filars. The ratio of filars between first and second filar arrays 282A, B include, but are not limited to 1:2, 1:3, 1:4, 2:3, 2:4, 2:5 and so on.

As shown in FIGS. 24 and 25A-B, in at least one embodiment, the aspiration sheath 260 includes a pull wire 280 anchored to the distal end of the sheath body 264 and extending toward the proximal end of the sheath body 264. In operation, the pull wire 280 is tensioned (e.g., from distal to proximal) to deflect the distal end of the sheath body 264. Actuation of the pull wire 280 angles the distal end of the sheath body 264, for instance to navigate the sheath body 264. In other examples, angling of the distal end of the sheath body enhances the direction of aspiration through the opening of the aspiration sheath 260, for instance toward collected thrombus material that has accumulated along one or more sides of the filter baskets 126, 226. In at least one embodiment, the distal end of the sheath body 264 includes a plurality of slits or relief cuts that enhance tip compliance and flexibility and thereby enhance direction of the aspiration catheter 260 with actuation of the pull wire 280. Optionally, the pull wire 280 is braced within the aspiration catheter 260 and axial loading (e.g., compression toward the distal end) of the pull write 280 permits actuation in a different orientation relative to tension.

As shown in FIG. 26, in at least one embodiment, the aspiration sheath 260 is delivered to the thrombus over a guidewire (such as the positioning guidewire 102) or over the shaft of the filter catheter 222. In one example a dilator 290 is inserted through the aspiration sheath 260, and as shown in FIG. 26, a dilator head 292 at least partially protrudes from the mouth of the aspiration sheath 260. The dilator includes the dilator head 292 and a shaft 294 coupled with the dilator head 292. As illustrated in FIG. 26, the dilator head 292 is optionally tapered from an outer diameter approximating the opening of the sheath body 264 to a smaller diameter (e.g., the dilator head 292 is tapered). In one embodiment, the dilator head 292 includes one or more relief scallops 296 (e.g., cuts, grooves, corrugations or the like) that permit fluid to flow past the dilator head 292 and into the aspiration sheath 260. The dilator head 292 and the one or more relief scallops 296 minimize vacuum sealing between the dilator head 292 and the aspiration sheath 260, and minimize air entrainment or air bubble retention in the aspiration sheath 260 for example as the dilator head 292 is moved through the lumen 266 of the sheath body 264 and the lumen 266 is at least partially filled with blood or saline. The inner diameter of the lumen 266 approximates the outer diameter of the dilator head 292 to minimize the edge of the sheath body 264 standing proud relative to the dilator head 292. In another example, the tapered profile of the dilator head 292 minimizes the stepped interface between the dilator head 292 and the sheath body 264 and accordingly provides for a flush (including near flush) transitions that minimizes snagging of vasculature, thrombus or the like.

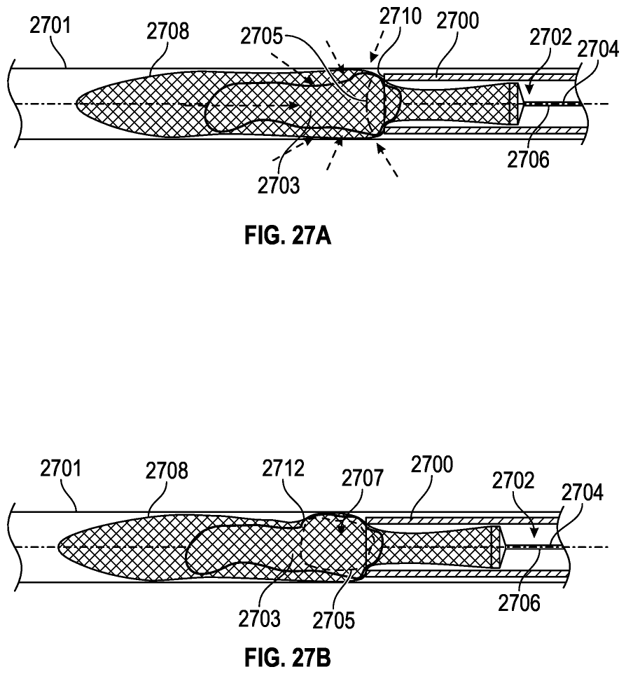

FIGS. 27A and 27B show another example of a collection catheter 2700 and a basket catheter 2704 in a vessel 2701. The basket catheter 2704 includes a basket 2708 coupled with a casket shaft 2706, and the basket shaft is slidably received within a catheter lumen 2702 of the collection catheter 2700. As shown in FIG. 27A, a portion of the basket 2708 is distal to a catheter mouth 2710 of the collection catheter 2700, and the thrombus 2703 is within the basket 2708.

The thrombus 2703 may, in some examples, have varied consistencies, density or the like. Some portions have a gelatinous consistency, other portions are fibrous and have an organized consistency, and still other portions have consistencies therebetween. Thrombus 2703 in some examples has mixed consistencies and accordingly behaves in sometimes unpredictable fashions.

The basket catheter 2704 is proximally moved relative to the collection catheter 2700. The relative movement of the basket 2708 pulls the basket and the thrombus 2703 toward and into the catheter mouth 2710. As shown in FIG. 27A, the thrombus 2703 has begun to lodge within the catheter mouth 2710 and is forming a thrombus plug 2705. Continued movement of the basket catheter 2704 with the basket shaft 2706 pulled relative to the collection catheter 2700 drives the thrombus 2703 toward the catheter mouth 2710, see FIG. 27B. The thrombus plug 2705 resists the proximal movement, and instead the thrombus 2703 aggregates proximate to the catheter mouth 2710. As shown in FIG. 27B, the thrombus plug 2705 enlarges with the proximal movement of the basket catheter 2704 into the collection catheter 2700. The enlarged thrombus plug 2705 aggravates the movement of the thrombus 2703 into the collection catheter 2700.

As further shown in FIG. 27B, the thrombus plug 2705 in some examples binds with the basket 2708 of the basket catheter 2704. Accordingly, as the basket 2708 is proximally pulled into the collection catheter 2700 the thrombus plug 2705 contorts, deforms or the like and the bound basket 2708 (e.g., snagged with the thrombus 2703) similarly contorts or deforms. In the example shown in FIG. 27B at a snagged basket portion 2712 of the basket 2708 has deformed and wrapped around the thrombus plug 2705. The binding of the basket 2708 and the thrombus 2703 further aggravates the plugging of the catheter mouth 2710 and thereby aggravates retraction of the thrombus 2703 into the collection catheter 2700 (e.g., the catheter lumen 2702).

In one example, a technician may attempt to pull the basket catheter 2704 to overcome the thrombus plug 2705 and the potentially snagged basket portion 2712. Increased proximal force, in some examples, further plugs the thrombus 2703 proximate to the catheter mouth 2710. Additionally, snagging of the of collection basket 2708 is further aggravated in some examples. In other examples proximal movement may pull the thrombus 2703, at least partially, into the collection catheter lumen 2702. However, the relative movement between the thrombus 2703 and basket 2708 relative to the catheter mouth 2710 may shear thrombus particulate from the thrombus 2703 (with the catheter mouth) and further complicate the procedure (e.g., require lytic infusions, additional retrieval procedures or the like). The proximal movement may, in some examples, damage one or more of the basket catheter 2704, the collection catheter 2700, or both and frustrate performance of the procedure. If the thrombus 2703 is not withdrawn, and the collection catheter 2700 and the basket catheter 2704 are not readily recoverable a procedure, such as a venous cutdown procedure, is conducted to surgically open the vessel 2701 and retrieve the catheters 2700, 2704.

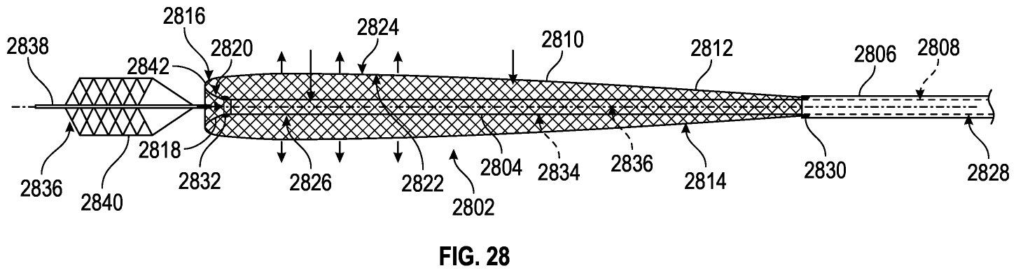

FIG. 28 illustrates an example of a thrombus capture and conveyor system 2800 that minimizes relative movement between the features of the system that engage with, capture, and withdraw thrombus from a vessel. Instead, the thrombus capture and conveyor system 2800 gathers thrombus proximate to a conveyor mouth 2818 of an everted thrombus conveyor 2810. Inversion of the thrombus conveyor 2810 shuttles thrombus in the manner of a conveyor carrying materials (e.g., the material is static relative to the conveyor, and moves with the conveyor) toward and into a conveyor sheath 2806. The thrombus is statically conveyed or shuttled with the thrombus conveyor 2810 and passively narrowed, thinned or the like as the thrombus conveyor 2810 travels toward the conveyor sheath 2806. At the conveyor sheath 2806 the thrombus and thrombus conveyor 2810 (static relative to the thrombus) are readily moved into the sheath without relative movement that causes plugging, snagging or the like in other systems.

As shown in FIG. 28, the thrombus capture and conveyor system 2800 includes a conveyor sheath 2806 having a sheath lumen 2808 as part of a conveyor catheter 2802. The conveyor catheter 2802 includes a conveyor shaft 2804 movably received within the sheath lumen 2808 of the conveyor sheath. As described herein, the thrombus conveyor 2810 is coupled with each of the conveyor shaft 2804 and the conveyor sheath 2806 of the conveyor catheter 2802. For instance, as shown in FIG. 28, a conveyor proximal end 2814 of the thrombus conveyor 2810 is coupled (welded, crimped, adhered or the like) at a conveyor sheath anchor 2830 of the conveyor sheath 2806. A conveyor distal end 2816 of the thrombus conveyor 2810 is coupled with the conveyor shaft distal portion 2826, for instance with a conveyor shaft anchor 2832.

The thrombus conveyor 2810 includes a conveyor substrate 2812 such as, but not limited to, a mesh, fibers, filaments, pliable elements or the like. The conveyor substrate 2812 captures and conveys thrombus while at the same time permitting the flow of liquids, such as blood. The thrombus conveyor 2810 including the substrate 2812 is pliable and configured to evert as shown in FIG. 28 (e.g., the everted or deployed configuration of the conveyor 2810). An exterior surface inversion 2842 is provided between the interior surface of 2822 of the conveyor 2810 and the exterior surface 2824 of the conveyor. As discussed herein, the exterior surface inversion 2842 is the transition location of the thrombus conveyor 2810 as the conveyor 2810 everts, for instance to engage with and shuttle thrombus into the thrombus conveyor 2810 and shuttle the thrombus toward the conveyor sheath 2806. A conveyor mouth 2818 of the thrombus conveyor 2810 extends from the exterior surface inversion 2842 toward the conveyor shaft distal portion 2826 (where the conveyor 2810 couples with the portion 2826).

Referring again to FIG. 28, the sheath lumen 2808 of the conveyor sheath 2806 (a component of the conveyor catheter 2802) receives a basket catheter 2836. As further shown in FIG. 28, the conveyor shaft 2804 (also of the conveyor catheter 2802) is received within the sheath lumen 2808 and includes its own conveyor shaft lumen 2834. The basket catheter 2836 is, in this example, provided within the conveyor shaft lumen 2834 of the conveyor shaft 2804. Accordingly, the basket catheter 2836 is nested within the conveyor shaft 2804 and the conveyor sheath 2806. The basket catheter 2836 extends through the thrombus conveyor 2810 coupled between the conveyor sheath 2806 and the conveyor shaft 2804.

As shown in FIG. 28, the basket catheter 2836 includes a basket shaft 2838 positioned within the conveyor shaft lumen 2834 of the conveyor shaft 2804. As shown, the at least one collection basket 2840 is coupled with the basket shaft 2838 and is provided in a deployed configuration distal to the conveyor mouth 2818 of the thrombus conveyor 2810. In other examples a plurality of collection baskets 2840 are coupled with and deployed from the basket shaft 2838. The one or more collection baskets 2840 are constructed with deformable materials, such as shape memory alloys, that are biased toward deployment as shown in FIG. 28, for instance, to positions the baskets 2840 in proximity to vessel walls and to facilitate capture of thrombus. In another example, the basket catheter 2836 includes a pull wire, nested catheter, rod or the like configured to manipulate the collection basket 2840 between the deployed configuration shown and a retracted configuration to facilitate delivery in the conveyor catheter 2802 to a treatment location.

In operation, collection basket 2840 (or baskets) is distally delivered relative to target thrombus in a vessel. For instance, the collection basket 2840 is in a retracted configuration and delivered through the conveyor catheter 2802 or a delivery catheter. The collection basket 2840 is expanded to the deployed configuration, for example because of withdrawal of the delivery catheter or conveyor catheter 2802. The conveyor catheter 2802 is deployed from a delivery catheter proximal to the target thrombus. The collection basket 2840 is proximally withdrawn and guides the thrombus toward to the conveyor mouth 2818.

With the target thrombus captured between the conveyor mouth 2818 and the proximally withdrawn collection basket 2840 (still distal to the thrombus conveyor 2810) the basket catheter 2836 is anchored to the conveyor shaft 2804, for instance with a clamp, fastener or the like. Accordingly, translating movement of the conveyor shaft 2804 (relative to the conveyor sheath 2806) also moves the basket catheter 2836 and its collection basket 2840. For instance, the collection basket 2840 is static relative to the conveyor shaft 2804 and the interior surface of 2822 of the thrombus conveyor 2810 coupled with the conveyor shaft 2804. The thrombus is guided between the basket 2840 and the thrombus conveyor 2810 toward the conveyor mouth 2818.

As shown in FIG. 28, the conveyor mouth 2810 extends from the exterior surface inversion 2842 toward the conveyor shaft distal portion 2826. The inversion 2842 is a fold or joint of the exterior surface 2824 of the conveyor as it transitions from facing away from the conveyor shaft (everted) to facing the conveyor shaft 2804 (inverted), for instance with the conveying motion of the thrombus conveyor 2810. The conveyor mouth 2818 is pliable and compresses the thrombus in a compliant manner as the thrombus moves proximally with the conveyor substrate (e.g., each is translationally static relative to the other). Additionally, the conveyor mouth 2818 has a conveyor mouth profile (e.g., a pocket, recess, cavity, bowl or the like) that readily receives thrombus and tapers from the exterior surface inversion 2842 toward the conveyor shaft anchor 2832.

As described herein, the static interface between the thrombus and the conveyor 2810 minimizes shearing and lineal compression of thrombus that otherwise generates plugging. Proximal movement of the conveyor shaft 2804 and the basket catheter 2836 guides the thrombus further into the thrombus conveyor 2810 and at the same time inverts the everted portion of the thrombus conveyor 2810, the exterior surface 2824. The exterior surface 2824 of the thrombus conveyor 2810 inverts at the exterior surface inversion 2842 as the conveyor shaft 2804 is proximally withdrawn relative to the conveyor sheath 2806. As the collection basket 2840, thrombus, basket shaft 2838 (of the basket catheter 2836) and the conveyor shaft 2804 are proximally withdrawn the thrombus is static relative to at least the interior surface 2822 of the conveyor 2810 facing and engaging the thrombus. At the same time the exterior surface 2824 of the thrombus conveyor 2810 is inverting at the exterior surface inversion 2842 (proximate the conveyor mouth 2818) and transitioning to the interior surface 2822 of the conveyor engaged with the guided thrombus.

Continued proximal movement of the conveyor shaft 2804 relative to the conveyor sheath 2806 further inverts the thrombus conveyor 2810 as the thrombus is guided into the conveyor 2810. The thrombus is static relative to the conveyor 2810 and the collection basket 2840 (coupled to the conveyor shaft 2804) as the conveyor 2810 is inverted around the thrombus. The thrombus is gradually enveloped by the inverting thrombus conveyor 2810 and the collection basket 2840 is optionally enveloped if inversion of the conveyor 2810 continues to thereby enclose the thrombus.

The enclosed thrombus is radially compressed and optionally lengthened as the thrombus conveyor 2810 deflects inwardly and stretches as it approaches the conveyor sheath 2860. At the conveyor sheath 2860 the compressed thrombus is readily delivered into the sheath lumen 2808 (proximate to the conveyor sheath anchor 2830 or sheath mouth) without plugging.

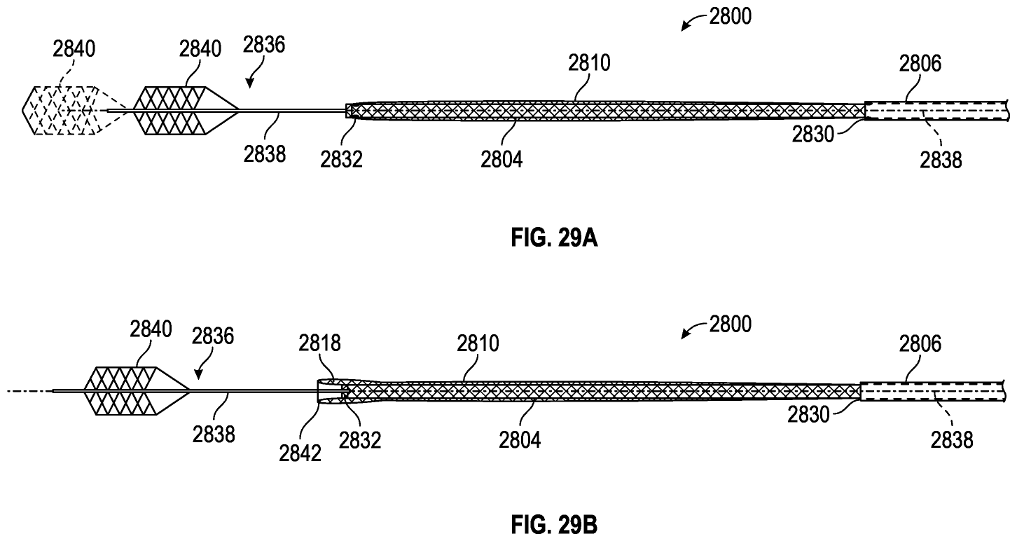

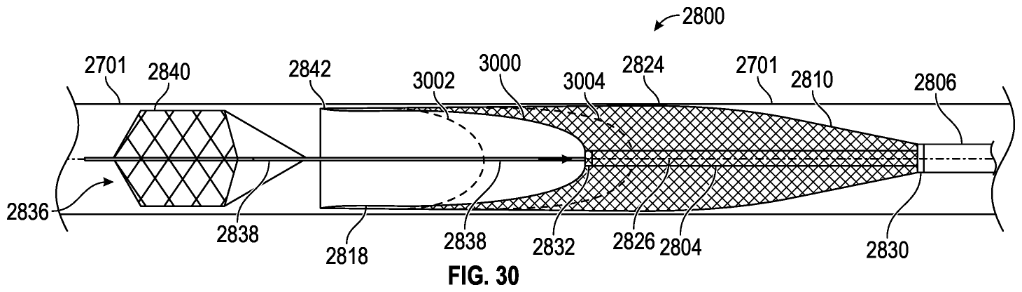

FIGS. 29A and 29B illustrate the thrombus capture and conveyor system 2800 with the thrombus conveyor 2810 in everted configurations. In the example shown in FIG. 29A, the thrombus conveyor is in a fully everted configuration, with the thrombus conveyor everted between each of the conveyor sheath anchor and the conveyor shaft anchor. An example basket catheter 2836 is shown distal to the thrombus conveyor 2810. The basket catheter 2836 includes at least one collection basket 2840. As shown in broken lines the basket catheter 2836 optionally includes multiple collection baskets 2840. As further discussed herein, the distal collection basket 2840 is optionally provided on a separate basket shaft 2838 to facilitate independent movement of the collection baskets 2840 (e.g., for separation of thrombus, and separate enveloping and shuttling with the thrombus convey 2810).

Referring again to FIG. 29A, as previously described the thrombus conveyor 2810 is an everted configuration, in this example a fully everted configuration. For instance, the conveyor shaft 2804 and conveyor sheath 2806 positioned as shown, and delivered through an introducer, delivery catheter or the like. As the conveyor shaft 2804 and sheath 2806 are deployed from the delivery catheter the thrombus conveyor is fully everted between the conveyor sheath anchor 2830 and the conveyor shaft anchor 2832. To begin inversion, the conveyor shaft 2804 is moved proximally relative to the conveyor sheath 2806. The movement forms the exterior surface inversion 2842 (see FIG. 28) as a portion of the thrombus conveyor 2810 proximate to the conveyor shaft anchor 2832 is inverted to the interior surface of the conveyor 2822 (FIG. 28).

FIG. 29B shows the thrombus conveyor 2810 optionally deployed in another everted configuration, for instance with a portion of the conveyor 2810 inverted at the exterior surface inversion 2842. In this example, the thrombus conveyor 2810 is delivered through an introducer or delivery catheter to a treatment location with the conveyor 2810 in the configuration shown (e.g., everted with a partial inversion). For instance, the conveyor shaft 2804 is proximally retracted relative to the conveyor sheath 2806 to initiate the exterior surface inversion 2842. The conveyor shaft 2804 is then optionally clamped to the conveyor sheath 2806 to maintain the thrombus conveyor 2810 in this everted configuration during delivery (e.g., through the vasculature, delivery catheter or the like). With the thrombus conveyor 2810 everted as shown a conveyor mouth 2818 is provided that extends from the exterior surface inversion 2842 toward the conveyor shaft anchor 2832.

FIG. 30 is a detailed view of the thrombus capture and conveyor system 2800 showing the conveyor mouth 2819 with various conveyor mouth profiles 3000 (solid lines), 3002, 3004 (dashed lines). The conveyor mouth 2818 extends from the exterior surface inversion 2842 toward the conveyor shaft distal portion 2826. The inversion 2842 is a fold or joint of the thrombus conveyor 2810 as it transitions from facing away from the conveyor shaft 2804 (everted) to facing the conveyor shaft 2804 (inverted). As discussed herein, proximal movement of the conveyor shaft 2804 relative to the conveyor sheath 2806 transitions the thrombus conveyor 2810 between the everted and inverted configurations and at the same time shuttles thrombus (and a captured collection basket) toward the conveyor sheath 2806.

In one example, with at least portions of the thrombus conveyor 2810 inverted and everted the conveyor exterior surface 2824 is directed away from the conveyor shaft. A bias is optionally applied to the exterior surface 2824 to bias it outwardly and into conformance with the vessel 2701 through one or more of the conveyor substrate materials, construction of the conveyor (e.g., braid pattern) or the inversion of the conveyor (e.g., transition between the everted and inverted configurations). The biased thrombus conveyor 2810 pliably conforms to the contours of the vessel 2710 and thereby provides a similarly conforming exterior surface inversion 2842 and (a proximate portion of the) conveyor mouth 2818 extending from the inversion 2842. The conforming shape of the conveyor 2810, inversion 2842 and the conveyor mouth 2818 facilitate the capture of thrombus and minimizes the escape of particulate around the conveyor 2810.

A plurality of example conveyor mouth profiles 3000, 3002, 3004 are shown in FIG. 30. The conveyor mouth profile includes one or more of shape, size, depth or the like of the thrombus conveyor 2810 configured to receive thrombus. In one example, the mouth profile 3000 is modulated through movement of the conveyor shaft 2804 (e.g., proximal relative to the conveyor sheath). The movement, as shown in FIG. 30, transitions the mouth profile between one or more configurations such as profiles 3000, 3002, 3004 as well as additional profiles with varied movement of the shaft 2804. Example mouth profiles 3000, 3002, 3004 are shown with solid and dashed lines in FIG. 30. For instance, the mouth profiles of the conveyor 2818 are shaped with a clinician specified depth, shape or the like while the collection basket 2840 remains deployed away from the thrombus conveyor 2810 (e.g., while the conveyor shaft 2804 is moved). After setting of the mouth profile to a specified shape the collection basket 2840 is moved proximally toward the thrombus conveyor 2810 with thrombus therebetween. In one example, with a relatively deep mouth profile 3000-3004 the reception of thrombus is facilitated within the thrombus conveyor 2810. Optionally, the collection basket 2840 is fixed at an offset distance (described herein) to minimize axial compression of the captured thrombus, and the thrombus conveyor 2810 is then inverted as discussed herein to convey the thrombus toward the conveyor sheath 2806. The collection basket 2840 moves with the conveyor shaft 2804 based on selective clamping between the basket and the shaft and the basket 2840 continues to guide thrombus into the thrombus conveyor 2810 for shuttling to the conveyor sheath 2806.

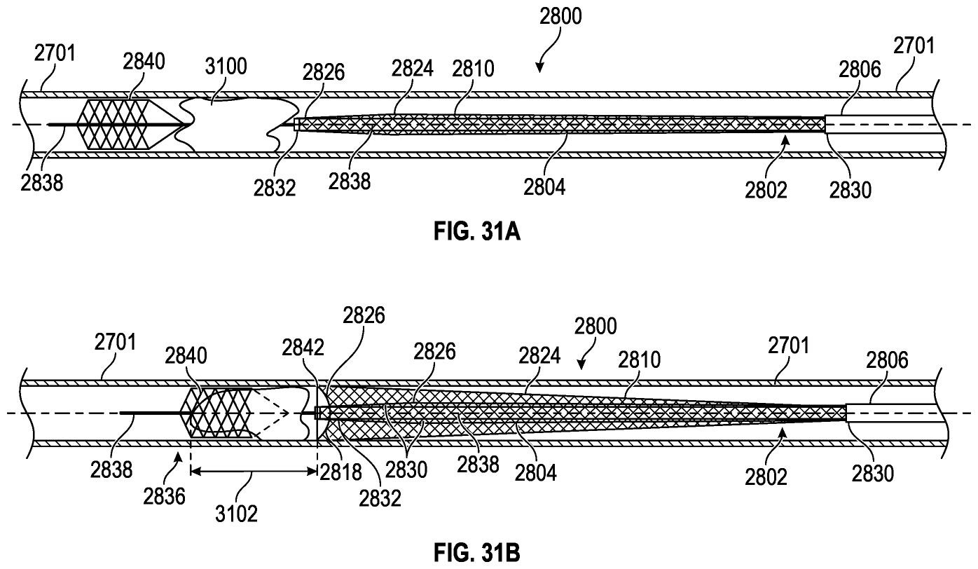

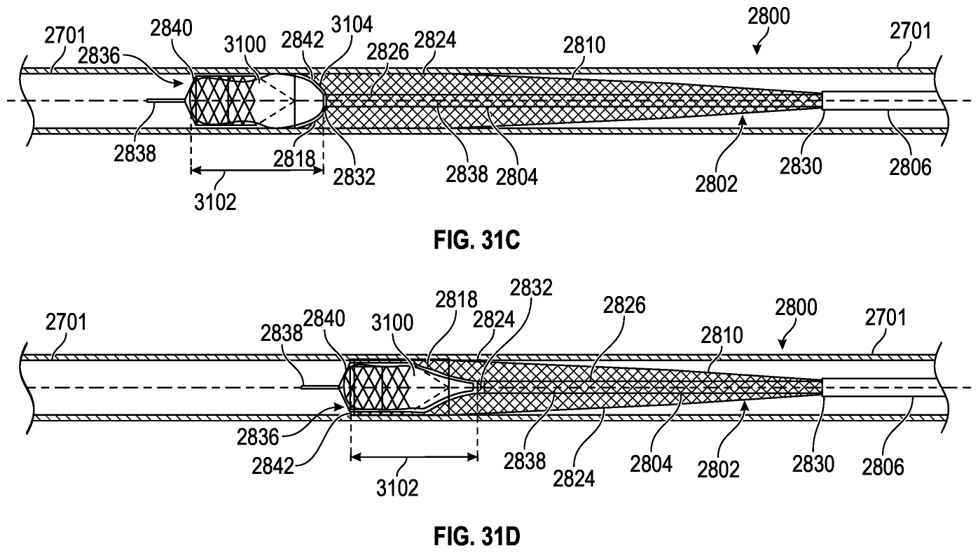

Referring now to FIG. 31A, the thrombus capture and conveyor system 2800 is shown with the thrombus conveyor 2810 initially everted (as shown in FIG. 29A). As shown in FIG. 29B the thrombus conveyor 2810 is then partially inverted with a portion of the exterior surface inverted to form the mouth profile 3000 of the conveyor mouth 2818 for reception of the 3100 therein. For instance, the conveyor catheter 2804 is moved proximally relative to the conveyor sheath 2806 to initiate folding inversion of the thrombus conveyor 2810 at the exterior surface inversion 2842 (see FIG. 31B).

The thrombus 3100 is between the collection basket 2804 and the thrombus conveyor 2810. For example, the collection basket 2840 is delivered within an associated sheath or catheter past the thrombus 3100 and then deployed. As discussed herein, the collection basket 2840 is in other examples deployed past a proximal portion of the thrombus, for instance within the thrombus itself (e.g., like an anchor) or as shown here with the basket deployed distal to the thrombus.

FIG. 31B shows the thrombus conveyor 2810 in an everted configuration, for instance with the conveyor mouth 2818 having a conveyor mouth profile 3104 provided with initiation of a partial inversion at the exterior surface inversion 2842. In one example, the partial inversion biases the conveyor exterior surface 2824 into intimate engagement along the vessel 2701 wall. In another example, the conveyor 2810 deploys by way of shape memory materials, biased movement of the conveyor substrate (e.g., a self-expanding braid) or the like into the deployed configuration. Inversion of the thrombus conveyor 2810 forms the conveyor mouth 2818, and optionally facilitates control of the conveyor mouth profile. As previously described, the conveyor shaft 2804 is moved relative to the conveyor sheath 2806 to control the profile of the conveyor mouth 2818 through inversion of the thrombus conveyor 2810.

In the configuration shown in FIG. 31B, the thrombus 3100 is captured between the collection basket 2840 and the thrombus conveyor 2810. For instance the deployed collection basket 2840 is proximally moved with its basket shaft 2838 toward the thrombus conveyor 2810 including the conveyor mouth 2818. The thrombus 3100 is captured without longitudinal compression (other than incidental compression as the basket 2840 and conveyor 2810 engage with the thrombus 3100) to minimize plugging, for instance when received at the conveyor sheath 2806. In one example, the collection basket 2840 and the conveyor mouth 2818 of the thrombus conveyor 2810 are arranged at an offset distance 3102 that corresponds to a length of the thrombus 3100 or the length between the conveyor 2810 and a proximal portion of the collection basket 2840, for instance viewed under fluoroscopy. In another example, the offset distance 3102 is set by a clinician based on feedback from the system 2800, for instance based on resistance (e.g., feel, haptic feedback or the like) to additional proximal movement of the basket catheter 2836 as the collection basket 2840 and thrombus conveyor 2810 capture the thrombus 3100 therebetween. The offset distance 3102 is fixed, for instance with a clamp that locks the basket catheter 2836 (e.g., the basket shaft 2838) with the conveyor catheter 2802 (e.g., the conveyor shaft 2804). Once locked, movement of either of the basket catheter 2836 or the conveyor catheter 2802 moves the collection basket 2840, thrombus 3100, and the inverted portion (e.g., interior) of the thrombus conveyor 2810 collectively, for instance, together without relative movement therebetween. The offset distance 3102 is maintained, and accordingly proximal movement of the collection basket 2840 guides the thrombus 3100 into the thrombus conveyor for shuttling by the conveyor 2810 as described herein.

FIG. 31C the thrombus conveyor 2810 is further inverted to shuttle the thrombus 3100 and the collection basket 2840 of the basket catheter fixed to the conveyor shaft 2804 of the conveyor catheter 2802 toward the conveyor sheath 2806. For instance the mouth profile of the conveyor mouth 2818 is enlarged (e.g., lengthened) to receipt the thrombus 3100 therein. As the thrombus 3100 is received along the inverted portion of the thrombus conveyor 2810 it engages with the conveyor 2810. With continued inversion of the conveyor 2810 the conveyor and the thrombus 3100 move together without relative movement therebetween. Instead, the thrombus conveyor 2810 moves the thrombus 3100 toward the conveyor sheath 2806 in the manner of a shuttle (conveyor belt or the like) transporting the thrombus 3100 toward the sheath.

As shown in FIG. 31C, the everted exterior surface 2824 remains engaged with the vessel 2701 wall even while the conveyor 2810 is inverted. The exterior surface inversion 2842 moves proximally with proximal movement of the conveyor shaft 2804, and the exterior surface 2824 folds or rolls inwardly (inverts to face toward the conveyor shaft 2804) while the remaining everted exterior surface 2824 is directed away from the conveyor shaft 2804 and toward the vessel 2701 wall.

With gradual inversion of the thrombus conveyor 2810 the thrombus 3100 remains captured between the collection basket 2840 and the conveyor 2810. In this example, the offset distance 3102 is maintained (e.g., with the conveyor shaft 2804 anchored to the basket shaft 2838), and accordingly the thrombus 3100 is maintained between the collection basket 2840 and the conveyor 2810 without axial compression therebetween. Instead, the thrombus 3100 is guided and engaged with the conveyor 2810 as the conveyor is inverted and conveyed toward the conveyor sheath 2806. As shown in FIG. 31C the thrombus 3100 is enveloped by the proximally moving inverted portion of the thrombus conveyor 2810 (e.g., the interior) and the thrombus 3100 and the thrombus conveyor 2810 move together toward the sheath 2806.

Referring now to FIG. 31D, the exterior surface 2824 of the thrombus conveyor 2810 continues to transition from facing outwardly to facing inwardly. As shown, the exterior surface 2824 continues to invert (e.g., roll, fold, peel or the like) at the exterior surface inversion 2842 to transition from facing away from the conveyor shaft 2804 to facing toward the conveyor shaft 2804. As the thrombus conveyor transitions from exterior to interior facing, the exterior surface 2824 peels away from the vessel 2701 wall.

The inverted thrombus conveyor 2810 surrounds the thrombus 3100 with inversion and conveys the thrombus 3100 proximally. The conveyor 2810 provides passive (radial) compression of the thrombus 3100 as the conveyor 2810 moves toward the conveyor sheath 2806 and tapers toward the opening of the conveyor sheath 2806. Because longitudinal compression is minimized (the thrombus may actually lengthen) the formation of thrombus plugs is minimized. Continued inversion of the thrombus conveyor 2810 conveys the captured thrombus 3100 toward the conveyor sheath 2806. The thrombus 3100 travels collectively with the thrombus conveyor 2810 (e.g., thrombus 3100 and conveyor 2810 are relatively static), and accordingly the thrombus experiences minimal shearing and longitudinal compression that may otherwise cause compression of the thrombus, snagging with the conveyor substrate, shearing of particulate from the thrombus 3100 or the like. Instead, the thrombus conveyor 2810 shuttles the captured thrombus toward the conveyor sheath 2806.

As further shown in FIG. 31D, in this example, the offset distance 3102 is maintained. The collection basket 2840 moves inside the thrombus conveyor 2810, is enveloped by the inverting conveyor 2810 and moves with the conveyor shaft 2804, thrombus 3100 and the inverted portion of the conveyor 2810 toward the conveyor sheath 2806.

At the distal end of the conveyor sheath 2806 the thrombus conveyor 2810 and the thrombus 3100 are slidably received within the sheath lumen 2808 (see FIG. 28). The conveyor catheter 2802 with the thrombus 3100 within the conveyor sheath 2806 is withdrawn through the vasculature to retract the thrombus from the treatment location. Optionally, aspiration pulls the thrombus 3100 from the thrombus conveyor 2810. In another example, the thrombus conveyor 2810 is withdrawn from a delivery catheter, cleaned of the thrombus 3100, and the conveyor 2810 is reset for additional thrombus removal. For example, the conveyor shaft 2804 is distally moved relative to the conveyor sheath 2806 to re-evert the thrombus conveyor 2810. The re-everted conveyor 2810 is then navigated to the treatment location (including a different treatment location) and the procedure described herein is repeated.

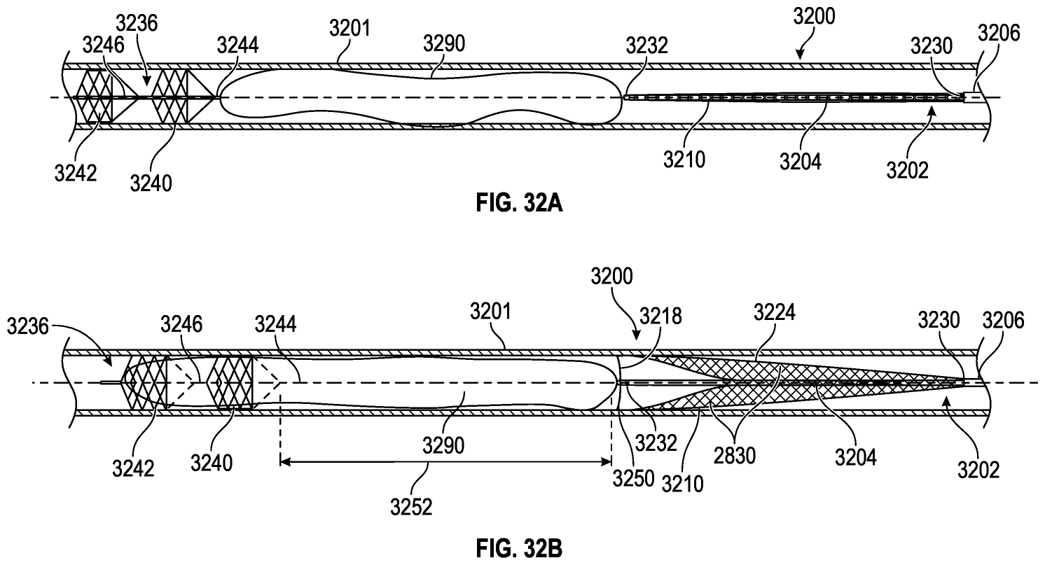

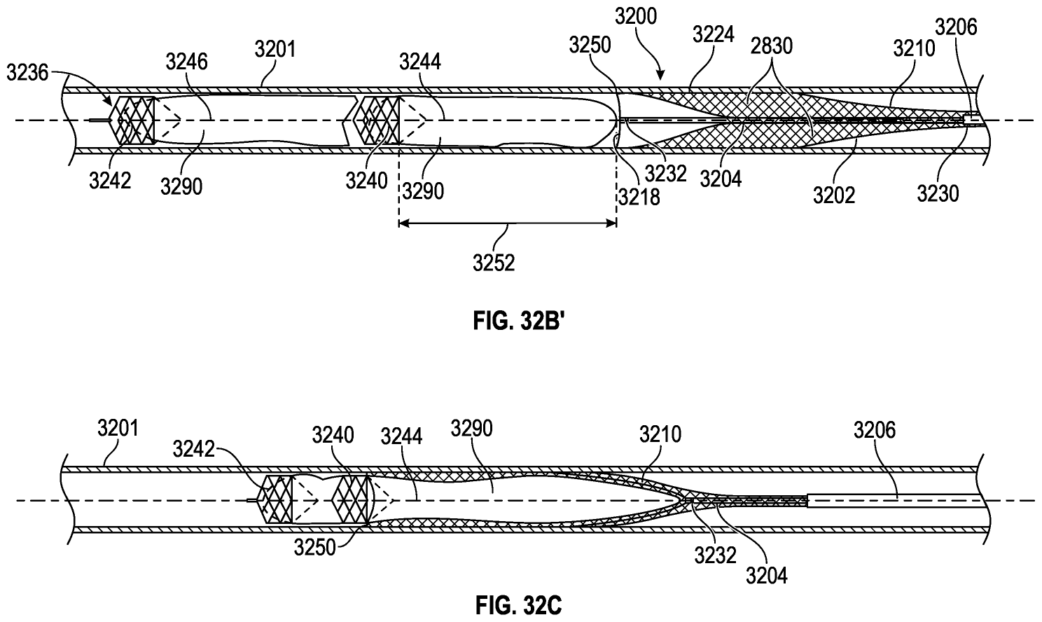

FIGS. 32A-32C show another example of a thrombus capture and conveyor system 3200. In this example the system 3200 shown in FIG. 32A includes a basket catheter 3236 having two or more collection baskets. As shown, the example catheter 3236 includes first and second collection baskets 3240, 3242, with the collection basket 3240 proximal relative to the basket 3242. The collection baskets 3240, 3242 are coupled along a basket shaft. In one example the basket shaft is a unitary shaft 3244 and coupled with each of the baskets 3240, 3242. This permutation is shown in FIG. 32B and permits the deployment of one or more collection baskets 3240 within the thrombus to anchor the basket catheter 3236 in the thrombus 3290 and facilitate guidance of the thrombus toward the thrombus conveyor 3210. In another example, the collection basket 3242 (the distal basket) is coupled with a component basket shaft 3246 nested within a component basket shaft 3244 having the collection basket 3240. Accordingly, the collection baskets 3240, 3242 and their associated shafts 3244, 3246 are movable relative to each other. As described herein, relative movement permits the separation (or partitioning) of the thrombus 3290 into smaller component thrombus for withdrawal from the vessel 3201.

Referring again to FIG. 32A, the thrombus 3290 is initially between the collection baskets 3240, 3242 and the thrombus conveyor 3210. For instance, the collection baskets 3240, 3242 are delivered within an associated sheath or catheter past the thrombus 3290 and then deployed. One or more of the collection baskets 3240, 3242 is deployed past a proximal portion of the thrombus and proximal to a distal portion of the thrombus, for instance within the thrombus 3290 itself (e.g., like an anchor), as shown in FIG. 32B below.

The thrombus conveyor 3210 is initially everted, and as shown in FIG. 32B partially inverted with a portion of the exterior surface inverted to form the mouth profile of the conveyor mouth 3218 for reception of the thrombus 3290 therein. For instance, the conveyor shaft 3204 is moved proximally relative to the conveyor sheath 3206 to deflect the thrombus conveyor 3210 and initiate inversion of the conveyor 3210 at the exterior surface inversion 3250 (see FIG. 32B). In one example, the inversion biases the conveyor exterior surface into intimate engagement along the vessel wall and minimizes the passage of thrombus past the conveyor 3210 while at the same time permitting fluid flow through the conveyor substrate (e.g., a mesh, filaments, braid or the like).

Referring now to FIG. 32B the first collection basket 3240 is deployed within the thrombus 3290. For instance, a sheath is withdrawn from the basket 3240 while positioned within the thrombus 3290. In one example, the collection basket 3240 provides an engagement feature, such as a cleat, barb, anchor or the like (including the basket itself) that engages with the thrombus 3290 and provides a robust coupling to facilitate guidance toward the thrombus conveyor 3210. In another example, the proximal collection basket 3240 separates the thrombus 3290 into component portions (see FIG. 32B′) to facilitate the conveyance of the component portions by the conveyor 3210.

Referring again to FIG. 32B, the second (distal) collection basket 3242 is deployed distally relative to the thrombus 3290. The second collection basket 3242 works in cooperation with the first collection basket 3240 to guide the thrombus 3290 toward the thrombus conveyor 3210. In an example like that shown in FIG. 32B′ the first collection basket 3240 separates the thrombus 3290 into component portions, and the second collection basket 3242 retains the second (distal) portion of the thrombus 3290 while the first (proximal) portion is conveyed with the conveyor 3210. The second collection basket 3242 then guides the second thrombus portion into the conveyor 3210.

FIG. 32B illustrates an example offset distance 3252. The offset distance 3252 corresponds to a length of the thrombus 3290 or the length between the conveyor 3210 and a proximal portion of the collection basket (the proximal collection basket 3240 in this example). Optionally, the system 3200 and thrombus 3290 are viewed under fluoroscopy to determine the offset distance 3252. In another example, the offset distance 3252 is set by a clinician based on feedback from the system, for instance based on resistance (e.g., feel, haptic feedback or the like). The offset distance 3252 is maintained between the thrombus conveyor 3210 and the collection basket 3240 (or baskets 3240, 3246), for instance with a clamp that locks the basket catheter 3236 (e.g., the collection shaft) with the conveyor catheter 3202 (e.g., the conveyor shaft). Accordingly, the basket 3240 (and option basket 3242) and the conveyor 3210 move collectively to guide the thrombus 3290 to the conveyor 3210 and initiate shuttling of the thrombus 3290 with the conveyor 3210.

As shown in FIG. 32C, the baskets 3240, 3242 cooperate with the thrombus conveyor 3210 to guide the thrombus 3290 toward the conveyor sheath 3206. The baskets 3240, 3242, coupled with the basket shaft 3244 move with the thrombus conveyor 3210 as the conveyor inverts from the everted configuration. As previously discussed, the baskets are fixed relative to the basket shaft 3244 to ensure the thrombus 3290 is guided toward the sheath 3206 while minimizing relative movement between the thrombus 3290 and the conveyor 3210 (that may cause shearing, plugging or the like). Instead, the thrombus 3290 is shuttled along with the moving thrombus conveyor 3210 toward the conveyor sheath 3206.

The thrombus conveyor 3210 inverts at the exterior surface inversion 3250 with the exterior surface of the conveyor 3210 transitioning from facing outwardly to facing inwardly. The exterior surface of the thrombus conveyor 3210 peels away from the vessel 3201 wall with inversion while the portion of the everted exterior surface proximal to the exterior surface inversion 3250 is coupled along the vessel wall. The exterior surface continues to invert (e.g., roll, fold, peel away from the vessel 3201 wall or the like) and transitions from facing away from the conveyor shaft 3204 to facing toward the conveyor shaft 3204. Continued inversion of the thrombus conveyor 3210 conveys the captured thrombus 3290 toward the conveyor sheath 3206.

The thrombus 3290 travels collectively with the thrombus conveyor 3210, and accordingly experiences minimal shearing that may otherwise cause compression of the thrombus, plugging, snagging with the conveyor substrate or the like. Instead, the inverted thrombus conveyor 3210 surrounds the thrombus 3290 and shuttles the thrombus proximally toward the conveyor sheath 3206. The conveyor provides passive compression of the thrombus as the conveyor moves toward the conveyor sheath. Because longitudinal compression is minimized (the thrombus may actually lengthen) the formation of the dense plugs is minimized.

Optionally, the offset distance 3252 (see FIG. 32B) is maintained. The collection baskets 3240, 3242 move inside the remaining everted portion of the thrombus conveyor 3210 while maintaining the distance 3252 between the basket 3240 and the conveyor shaft 3204 (e.g., the conveyor shaft anchor 3232 proximate to a distal portion of the conveyor shaft 3204). Accordingly, the thrombus 3290 is maintained between the baskets 3240, 3242 and the conveyor shaft 3204 without relative movement to the conveyor 3210. Instead, the thrombus 3290 is passively compressed and delivered toward the conveyor sheath 3206 along with the collection baskets 3240, 3242, the conveyor shaft 3204 and the inverted (interior facing) portion of the conveyor 3210.

In an example shown in FIG. 32B′, with the first collection basket 3240 movable relative to the second collection basket 3242, a first clamp locks the first collection basket 3240 (and its basket shaft 3244) to the thrombus conveyor 3210. The second collection basket 3242 and its basket shaft 3246 remain decoupled (and movable) relative to the conveyor 3210 and the first basket 3240. In this example, the second collection basket 3242 remains deployed with the partitioned thrombus portion captured with the basket 3242 to retain a second portion of the thrombus 3290 while the remainder of the system 3200 moves collectively relative to the second collection basket (and its associated basket shaft) to convey the first thrombus portion.

In one example, the basket shaft 3244 is fixed (e.g., clamped, locked or the like) with the thrombus conveyor 3210, for instance with the conveyor shaft 3204. As the conveyor shaft 3204 or the basket shaft 3244 is moved, for instance proximally, the other of the basket shaft 3244 (and its basket 3240) and the conveyor shaft 3204 moves in correspondence. As shown in FIG. 32B′, the offset distance 3252 is maintained between the basket 3240 and the conveyor mouth 3218 to ensure the thrombus 3290 (portion with the first collection basket 3240) is guided into the conveyor and shuttled by the conveyor 3210 toward the conveyor sheath 3206. The thrombus conveyor 3210 continues to invert (e.g., roll, fold, peel from the vessel wall or the like) and accordingly shuttles the thrombus 3290 into and through the conveyor sheath 3206. The second portion of the thrombus 3290 is retained with the collection basket 3242. By partitioning the thrombus 3290 the system 3200 is able to convey and deliver manageable portions of the thrombus 3290, for instance, with smaller thrombus conveyors 3210, baskets, conveyor catheter components (e.g., sheath 3206, shaft 3204) or the like.

Because the thrombus conveyor 3210 is coupled at its conveyor distal end with the conveyor shaft 3204 and at its conveyor proximal end is coupled with the conveyor sheath 3206 the conveyor 3210 is readily re-deployed (e.g. everted) to the configuration shown in FIG. 32B′ after having withdrawn the first basket 3240 with the first portion of the thrombus 3290. Re-deployment is conducted in an example without removing the conveyor 3210 from the vessel to manually evert the conveyor 3210. The second collection basket 3242 and remaining second portion of the thrombus 3290 are then guided toward the conveyor mouth 3218 in a similar manner to the first collection casket 3240 (e.g., with proximal movement of the basket shaft 3246). Optionally, once an offset distance 3252 is reached, the basket shaft 3246 is fixed to the conveyor shaft 3204, and the second collection basket 3242, the second portion of the thrombus 3290, and the thrombus conveyor 3210 move together to shuttle the thrombus into and through the conveyor 3210 as it inverts.

In another example, the thrombus capture and conveyor system 3200 includes the first collection basket 3240 without the second collection basket 3242. The first collection basket 3240 is deployed as shown in FIG. 32B′ to separate the thrombus 3290 and convey the first thrombus portion into and through the thrombus conveyor 3210, and the conveyor 3210 shuttles the thrombus into the conveyor sheath 3206. The basket shaft 3244 and the first basket 3240 are optionally withdrawn from the system 3200, for instance through the conveyor sheath 3206. In one example, the basket 3240 is cleaned (e.g., aspirated) to remove thrombus and is navigated in a delivery catheter, the conveyor sheath 3206 or the like to the treatment site for continued operation in a thrombectomy procedure. For instance, after removal of thrombus from the first basket 3240 (or use of a different basket) the basket 3240 is re-deployed distal to the second thrombus portion 3290, and the second portion is guided to the reset thrombus conveyor 3210 for shuttling of the second portion into the conveyor sheath 3206.

As noted herein above, because the thrombus conveyor 3210 is coupled with the conveyor shaft 3204 and the conveyor sheath 3206 the conveyor 3210 is readily re-deployed (e.g. everted) to the configuration shown in FIG. 32B′ after withdrawal of the first basket 3240 with the first portion of the thrombus 3290. With the thrombus conveyor 3210 re-deployed and the collection basket 3240 positioned distal to the remaining (second) portion of thrombus 3290, the capture and shuttling of the thrombus is repeated with inversion of the thrombus conveyor 3210.

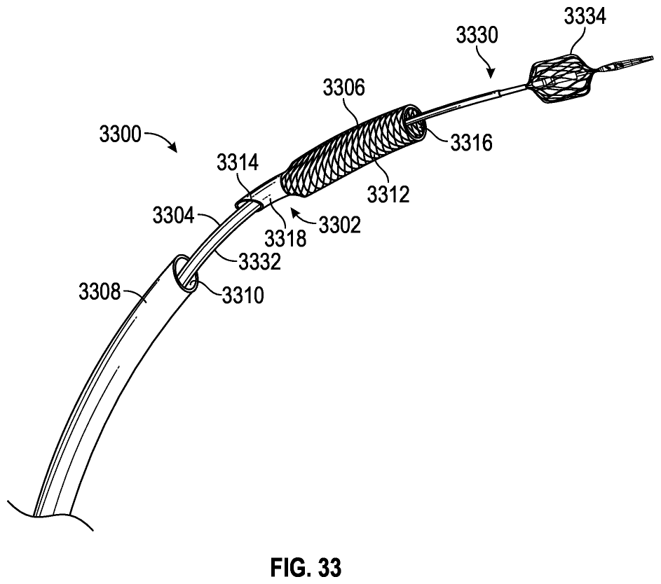

FIG. 33 illustrates another example of a thrombectomy system 3300. The system 3300 includes a trumpet catheter 3302 having a trumpet shaft 3304 coupled with a trump 3306. As shown in FIG. 33, the trumpet shaft 3304 in this example is a stylet, rail, guidewire or the like and has a minimized profile relative to a tubular catheter, sheath or the like. As discussed herein, the trumpet shaft 3304 (and the basket shaft of the basket catheter 3330) assume a minimal proportion of the sheath lumen 3310 of the trumpet sheath 3308 and thereby enhance the available remaining volume of the sheath lumen 3310 for additional catheters, instruments, passage of thrombus or the like.

Referring again to FIG. 33, the trumpet 3306 is constructed with a trumpet substrate 3312. The trumpet substrate 3312 captures thrombus, for instance delivered with the basket catheter 3330, while permitting fluid flow (e.g., blood or the like). The trumpet substrate 3312 includes, but is not limited to, fibers, filaments, elements or the like that are braided, meshed, knitted, non-woven or the like. As further shown in FIG. 33, the trumpet catheter 3302 includes a trumpet shaft anchor 3318 that couples the trumpet 3306 with the trumpet shaft 3304 (e.g., a guide wire, catheter shaft or the like). In this example, the trumpet shaft anchor 3318 includes a catheter port 3314 configured to receive catheters therein. The catheter port 3314 is proximate to a proximal end of the trumpet 3306 and receives catheters and other instruments to facilitate delivery of the catheters and instruments thereon through the trumpet mouth 3316. The catheter port 3314 and the trumpet shaft 3304 facilitate the decreased profile of the trumpet catheter 3302 (e.g., in contrast to a cylindrical catheter coupled with the trumpet 3306) and accordingly enhance available space within the sheath lumen 3310 of the trumpet sheath 3308. As shown in FIG. 33 the profile of the trumpet shaft 3304 and the adjacent basket shaft 3332 are readily received within the sheath lumen 3310 of the trumpet sheath 3308. The trumpet shaft 3304 and the basket shaft 3332 assume a minimal proportion of the sheath lumen 3310 and thereby enhance the remaining proportion of the sheath lumen 3310 for additional catheters, instruments, passage of thrombus or the like.

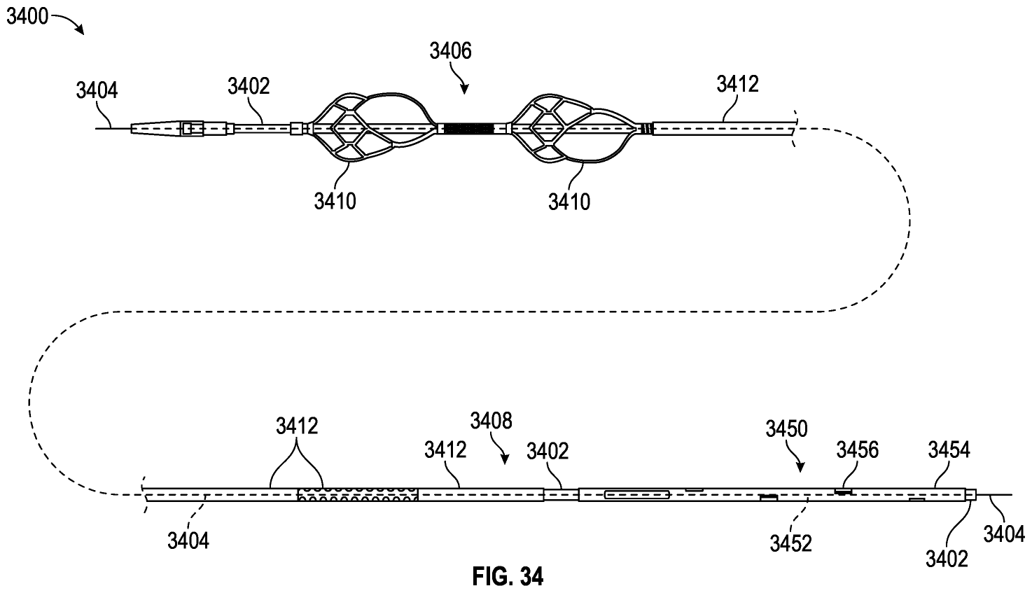

An example basket catheter 3330 is shown in FIG. 33. The basket catheter 3330 is similar in some regards to the basket catheters previously discussed herein. The basket catheter 3330 includes a basket shaft 3332 coupled with a collection basket 3334. The collection basket 3334 is delivered on the basket shaft 3332 through the trumpet 3306 to a treatment site. For instance, the basket shaft 3332 and the trumpet 3306 are delivered through the catheter port 3314 and through the trumpet mouth 3316 for deployment distal to thrombus with the trumpet 3306 proximal to thrombus (e.g., thrombus is between the trumpet 3306 and the collection basket 3334. In another example, the basket catheter 3330 and the trumpet catheter 3302 are loaded into a delivery catheter or the like together. For instance, the collection basket 3334 is pre-positioned within the trumpet 3306 and the basket 3334 and trumpet 3306 are loaded into the delivery catheter, and advanced through the delivery catheter to a treatment site. Delivery of the collection basket 3334 through a tubular trumpet catheter within the sheath lumen 3310 is thereby unnecessary. Accordingly, the sheath lumen 3310 includes a relatively large volume of available space for the delivery and movement of catheters, instrument, aspiration, infusion, withdrawal of thrombus or the like. FIG. 34 shows one example of a catheter system 3400 including a basket catheter 3402 and a basket sheath 3412 slidably positioned along the basket catheter 3402. As further shown, the basket catheter 3402 includes one or more baskets 3410. The one or more baskets 3410 include, for example, deployable baskets 3410 (e.g., self-deploying, actuated deploying or the like) configured to expand into the configuration shown in FIG. 34 with the filaments, elements, mesh or the like of the basket 3410 positioned radially away from the remainder of the basket catheter 3402. In operation, the basket sheath 3412 is moved proximately and withdrawn from over top of the baskets 3410 after positioning of the basket catheter 3402 at a position proximate to thrombus. For instance, the basket catheter distal end 3406 is positioned distal relative to thrombus and the basket sheath 3412 is then withdrawn to reveal the baskets 3410 and permit their deployment.

In one example, the catheter system 3400 includes a procedural guidewire 3404 as one example of a first catheter. The procedural guidewire 3404 is initially navigated to a target location within the vasculature and the procedural guidewire 3404 then serves as a rail for delivering features of the remainder of the catheter system 3400. For instance, the basket catheter 3402 (an example of a second catheter) is delivered over the procedural guidewire 3404 to position features of the basket catheter, such as the baskets 3410 proximate to the thrombus.

Referring now to another portion of the catheter system 3400 shown in FIG. 34, the catheter system 3400 includes a basket catheter proximal end 3408. As shown, the basket sheath 3412 extends over the basket catheter 3402 to permit manipulation of the basket sheath 3412, for instance, to reveal the basket catheters 3410 as previously described. As further shown in FIG. 34, the procedural guidewire 3404 extends through a lumen of the basket catheter (in one example, a second catheter lumen) and extends proximally relative to the remainder of the basket catheter 3402 to permit movement of the basket catheter 3412 along the procedural guidewire 3404 while the procedural guidewire 3404 is held static, for instance, by a clinician or the like during the procedure.

As further shown in FIG. 34, one example of a locking mechanism 3450 is provided with the second catheter such as the basket catheter 3402. The locking mechanism 3450, as described herein, provides a cooperative fitting configured to lock or statically couple the basket catheter 3402 (a second catheter) with a first catheter. One example of a first catheter is the procedural guidewire 3404 also shown in FIG. 34. As described herein, the locking mechanism 3450 includes a locking element, such as an actuator collar 3454 and a locking collet interior to the actuator collar 3454. The actuator collar 3454 is movably coupled with the locking collet.

The actuator collar 3454 includes one or more actuator bosses 3456 that are selectively aligned with corresponding locking struts of the locking collet to statically couple (e.g., lock, retain, hold, anchor or the like) the basket catheter 3402 with the procedural guidewire 3404. For instance, as described herein, the alignment of one or more of the actuator bosses 3456 with correspondingly locking struts of the locking collet deflects the locking struts and biases the locking struts into a second catheter lumen of the basket catheter 3402. The procedural guidewire 3404, an example of a first catheter, is received within the second catheter lumen of the basket catheter 3402. The deformed locking struts biased into the deformed configuration by the actuator bosses 3456 engage with the first catheter, such as the procedural guidewire 3404, and thereby anchor the basket catheter 3402 to the procedural guidewire 3404 based on deflection and engagement of the locking struts with the procedural guidewire 3404. This configuration is referred to herein as a locked configuration.

In the locked configuration, the deformed locking struts fix the basket catheter 3402 and the received instrument such as the procedural guidewire 3404 together and minimize relative movement therebetween. Instead, a clinician may hold the procedural guidewire 3404 while navigating other instruments such as a retrieval catheter or the like over the basket catheter 3402. Because of the locking mechanism 3450 in the locked configuration, navigating movement of the trumpet catheter or other instruments over top of the basket catheter 3402 does not cause incidental movement of the procedural guidewire 3404 or the basket catheter 3402 relative to the guidewire 3404. Instead, the locking mechanism 3450 anchors the basket catheter 3402 to the held procedural guidewire 3404 to prevent (e.g., minimize or eliminate) movement of the basket catheter 3402. In another example, a clinician may initiate movement of the basket catheter 3402 with cooperative movement of the procedural guidewire 3404 statically coupled with the basket catheter 3402. For instance, movement of the procedural guidewire 3404 is transmitted to the basket catheter 3402 through the locking mechanism 3450 in the locked configuration.

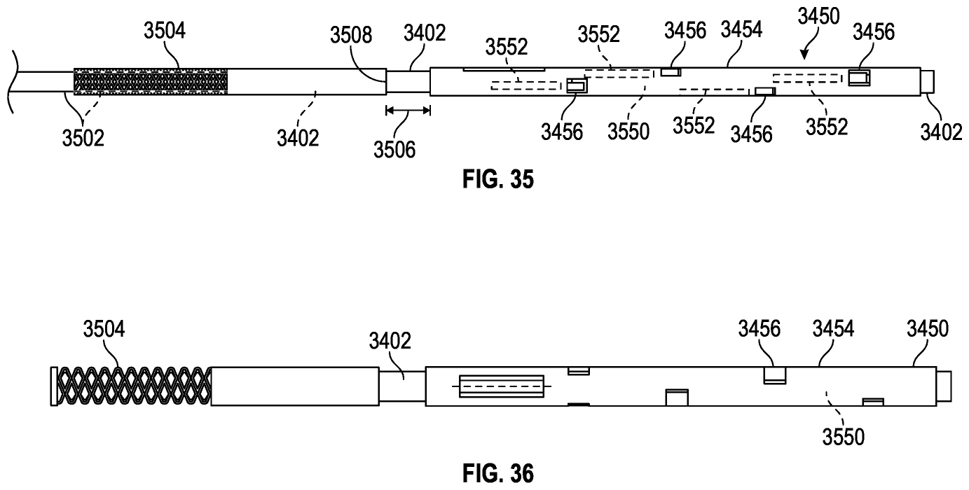

FIG. 35 shows a detailed view of a proximal portion of the basket catheter 3402. A base shaft 3502 of the basket catheter 3402 interfaces with one or more other features of the basket catheter 3402 including, but not limited to, the locking mechanism 3450 previously described and shown in FIG. 1. In the example shown in FIG. 35, the basket catheter 3402 includes an optional basket catheter interface 3504 that receives a portion of the basket catheter such as the base shaft 3502 and couples with the remainder of the basket catheter 3402. The basket catheter interface 3504, in one example, includes a mesh, braided feature or the like that transitions to the remainder of the basket catheter 3402 including, in this example, the locking mechanism 3450. In one example, the base shaft 3502 of the basket catheter 3402 is received in the basket catheter interface and thereby interconnects the distal portions of the basket catheter 3402 with the proximal portions of the basket catheter such as the locking mechanism 3450. Optionally, the basket catheter interface 3504 is mechanically deformed, adhered, welded or the like to the base shaft 3502 of the basket catheter 3402 to couple the base shaft 3502 with the remainder of the basket catheter 3402 including, for example, the locking mechanism 3450.

The locking mechanism 3450 is shown in further detail in FIG. 35. In this example, the locking mechanism 3450 includes the actuator collar 3454 moveably coupled along the locking collet 3550. As previously described, the actuator collar 3454, is a movable component that is movable over the basket catheter 3402, for instance, over the locking collet 3550. As previously described, the movement of the actuator collar 3454 relative to the locking collet 3550 is conducted by way of one or more of longitudinal movement, rotational movement or the both between the collar and the collet. This movement aligns one or more actuator bosses 3456 of the actuator collar 3454 with corresponding locking struts 3552 of the locking collet 3550. The locking struts 3552 are shown with dashed lines in FIG. 35. In an example, the actuator bosses include, but are not limited to, one or more flanges, tines, prongs, cams, ridges, feet or the like. In one example, the one or more actuator bosses 3456 have an interior profile that extends into a collar lumen of the actuator collar 3454, for instance, to engage and deform components of the locking collet 3550 within the collar lumen, such as the locking struts 3552. Deformation of the locking struts 3552 when engaged by the actuator bosses 3456 statically couples the first and second instruments (e.g., the procedural guidewire and basket catheter) together thereby locking, anchoring, holding, retaining or the like, the basket catheter 3402 to the procedural guidewire 3404.

As further shown in FIG. 35, by way of dashed lines in the figure, one or more locking struts 3552 are provided with the locking collet 3550. In one example, the locking struts include, but are not limited to, deformable elements that are deflected by one or more of the actuator bosses 3456. Inward deflection of the locking strut 3552 engages the locking strut against an instrument within a collet lumen of the locking collet 3550. In one example, the collet lumen of the locking collet is coextensive with or includes the second catheter lumen of the basket catheter 3402. Inward deflection of one or more locking struts 3552 engages the deflected locking struts 3552 with an instrument, such as the procedural guidewire 3404, and locks the instrument to the locking mechanism 3450. The procedural guidewire 3404 is locked or statically coupled with the second catheter, in this example the basket catheter 3402, thereby statically coupling the basket catheter 3402 with the procedural guidewire 3404 and limiting relative movement therebetween.

As further shown in FIG. 35, in one example, an actuation span 3506 is provided between the actuator collar 3454 and the remainder of the basket catheter 3402. In one example, the actuator span 3506 is used by the clinician to confirm positioning of the actuator collar 3454 in the locking configuration. For instance, as the actuator collar 3454 is moved into the locked configuration (e.g., toward a catheter shoulder 3508), the actuator collar 3454 covers the actuator span 3506. Covering the actuation span 3506 is visually, haptically or both, in one example is an indicator for the clinician that the actuator collar 3454 is moved to a locked position and the basket catheter (e.g., an example of a second catheter) is statically coupled to the procedural guidewire (e.g., an example of a first catheter). In another example, the engagement of the actuator collar 3454 with the catheter shoulder 3508 provides a haptic or tactile sensation to the clinician operating the locking mechanism 3450 indicative of the actuator collar 3454 having been moved into the locked position.

FIG. 36 is another detailed view of a portion of the basket catheter 3402. In this example, the base shaft 3502, previously shown in FIG. 35, is removed thereby revealing the basket catheter interface 3504 including, for instance, a mesh, laser cut array of filaments or the like configured to receive and couple with the base shaft 3502 of the catheter.

As further shown in FIG. 36, the locking mechanism 3450 including the actuator collar 3454 is shown again. The actuator collar 3454, as previously described, includes one or more actuator bosses 3456. In one example, the actuator bosses 3456 are staggered or positioned around the actuator collar 3454 in a manner that coincides with one or more of the locking struts 3552 of the locking collet 3550. With the actuator bosses 3456 positioned in these locations, movement of the actuator collar 3454, for instance, distally to the locked position correspondingly moves one or more of the actuator bosses 3456 into alignment with one or more of the locking struts 3552. This movement of the actuator bosses 3456 into alignment with one or more locking struts 3552 thereby ensures corresponding locking between the basket catheter 3502 (an example second catheter) and the procedural guidewire 3404 (an example first catheter) housed therein.

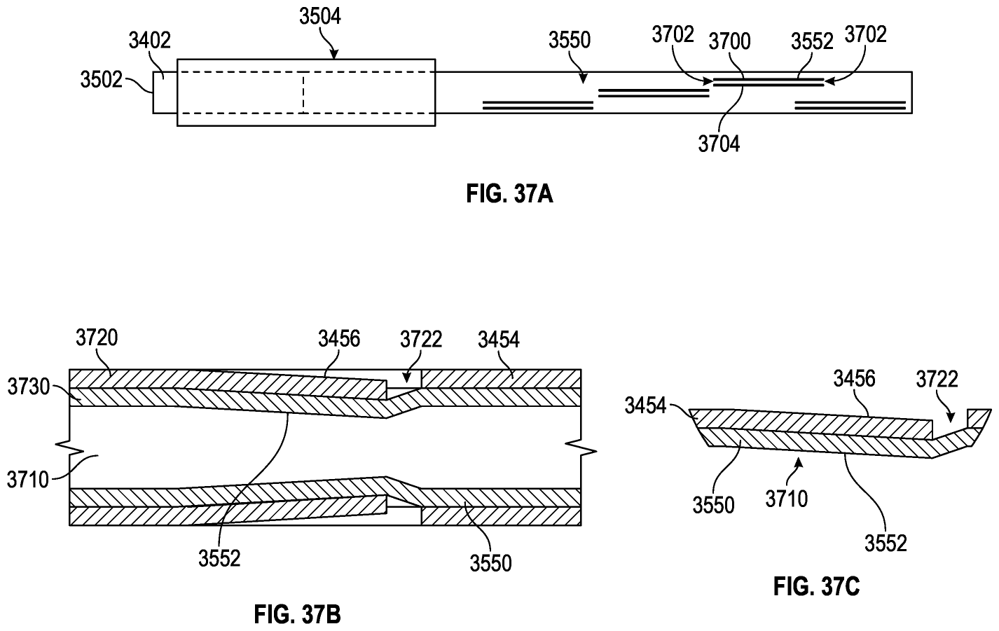

FIG. 37A is a schematic view of a portion of the catheter system 3400 previously shown in FIG. 34 with the actuator collar 3454 removed thereby revealing the locking collet 3550 and its locking struts 3552. The base shaft 3502 of the basket catheter 3402 is also shown coupled with the basket catheter interface 3504. The locking collet 3550 is, in one example, a portion of the second catheter such as the basket catheter 3402 previously described and shown herein. In another example, the locking collet 3550 is coupled with an instrument such as the second catheter. For instance, the basket catheter interface 3504 serves as an intermediate or interface from the locking mechanism including the locking collet 3550 to the remainder of the basket catheter 3402.

As shown, the locking collet 3550 includes a plurality of locking struts 3552. The locking struts 3552, in one example, include one or more strut members 3700 as portions of the sidewall of the basket catheter 3402. In one example, the strut members 3700 are isolated from other portions of the second catheter, such as the basket catheter 3402, with one or more slits 3704 provided along portions of the locking strut 3552. In one example, the slits 3704 isolate portions of the locking struts 3552 from the remainder of the catheter 3402 to permit selective deflection of the strut members 3700 to engage the deflected strut members 3700 with one or more other instruments such as the first catheter, or a procedural guidewire 3404, received within the basket catheter 3402.

In the example shown in FIG. 37A, the strut members 3700 are elongate and extend from a first strut end 3702 to a second strut end 3702. The slits 3704 are provided on either side of the strut member 3700 and accordingly isolate the strut member 3700 between the strut ends 3702 to facilitate the deflection of the locking strut 3552 while the remainder of the basket catheter 3402 is isolated from deflection caused by the actuator bosses of the actuator collar 3454. Instead, the locking struts 3552 preferentially bias or deflect relative to the remainder of the basket catheter 3402 to engage with one or more instruments provided within the basket catheter 3402 such as the procedural guidewire 3404 previously described and shown herein.

As further shown in FIG. 37A, the locking struts 3552, in this example, are provided in a staggered configuration around the locking collet 3550. In one example, the locking struts 3552 are arranged in the staggered configuration to ensure engagement between one or more of the locking struts 3552 and the actuator bosses 3456 of the actuator collar 3454. As the actuator collar 3454 is moved into the locked position (distally in this example) one or more of the actuator bosses 3456 align with one or more locking struts 3552 to transition the locking mechanism 3450 into the locked configuration. In one example, the actuator bosses 3456 have a complementary orientation or configuration that matches or is similar to the configuration of the locking struts 3552. Accordingly, as the actuator collar 3454 is moved to the locked position, the actuator bosses 3456 align with and deflect correspondingly associated locking struts 3552. In another example, the locking struts 3552 and actuator bosses 3456 have cooperative alignments, for instance, alignments that do not match but ensure one or more of the locking struts 3552 are aligned with one or more actuator bosses 3456 upon movement of the actuator collar 3454 to the locked position. For instance, as shown in FIG. 37A, in this example, the locking struts 3452 have a staggered configuration with the locking struts 3552 having a generally helical arrangement around the locking collet 3550. In another example, the actuator bosses 3456 have a converse pattern, for instance, an opposed helical pattern so that the locking struts 3552 have a clockwise helical pattern while the actuator bosses 3456 have a counterclockwise helical pattern. With the clockwise and counterclockwise converse configurations one or more of the locking struts 3552 is ensured to align with one or more of the actuator bosses 3456 as the actuator collar 3454 is positioned in the locked position to transition the locking mechanism 3450 to the locked configuration.

In another example, the locking struts 3552 and the actuator bosses 3456 of the respective locking collet 3550 and actuator collar 3454 are arranged in a staggered configuration, for instance, with the locking struts 3552 at an interval of approximately every 15 degrees and the actuator bosses 3456 at a different interval, for instance, an interval of 5, 20, 25, 30 degrees or the like. In one example, the frequency or interval of the actuator bosses 3456 is greater than that of the locking struts 3552. In yet another example, the interval frequency of the locking struts 3552 is greater than that of the actuator bosses 3456. Accordingly, with one or more of these staggered configurations, at least some of the actuator bosses 3456 and the locking struts 3552 are ensured to overlie each other and have a coincident relationship wherein one or more of the actuator bosses 3456 is aligned with one or more of the locking struts 3552 no matter how the locking collet 3550 is moved to the locked position. For instance, with one or more of rotation or translational positioning, the actuator collar 3454 is ensured to have one or more of its actuator bosses 3456 overly one or more of the locking struts 3552. Deflection of one or more locking struts 3552 is ensured thereby ensuring the locking mechanism 3450 transitions to the locked configuration and statically couples the first instrument with the second instrument (e.g., in one example, the procedural guidewire 3404 with the basket catheter 3402).

FIGS. 37B and 37C show a portion of the locking mechanism 3450 in the locked configuration including detailed examples of one or more of the actuator bosses 3456 of the actuator collar 3454 deflecting one or more of the locking struts 3552. Referring first to FIG. 37B, actuator bosses 3456 are shown overlying locking struts 3552. For instance, the actuator bosses 3456 as one or more of tines, flanges, ridges or the like are shown in a deflected configuration and correspondingly deflect the underlying locking struts 3552. The deflected locking struts 3552 are shown in a deflected configuration with the locking struts 3552 extending into a second catheter lumen 3710. In one example, the second catheter lumen 3710 is a lumen of a basket catheter, such as the basket catheter 3402. The deflected locking struts 3552 are received in the second catheter lumen 3710 and engage with one or more instruments provided in the second catheter lumen 3710. In one example, the instrument received in the second catheter lumen 3710 includes, but is not limited to, a first catheter such as a procedural guidewire 3404.

As further shown in FIGS. 37B and 37C, the actuator boss 3456, in this example, is a flange, tine, projection or the like, for instance, isolated from the remainder of the actuator collar 3454 by one or more slits 3722. The slits 3722 isolate the actuator boss 3456 and permit the selected deflection of the actuator boss 3456, for instance, by way of plastic deformation or the like. With movement of the actuator collar 3454 to align the one or more actuator bosses 3456 with the locking struts 3552, the locking strut 3552 show in FIGS. 37B and 37C is deflected as shown.

FIG. 37C shows a detailed example of the deflection of one of the locking struts 3552 previously described herein. For instance, the actuator boss 3456 has deflected the locking strut 3552 into the second catheter lumen 3710 previously shown in FIG. 37B. The locking strut 3552, when deflected, engages with an instrument such as the first catheter or procedural guidewire 3404 received in the second catheter lumen 3710. The locking strut 3552 engaged with the procedural guidewire 3404 statically couples the procedural guidewire to the locking collet 3550 and accordingly statically couples the procedural guidewire 3404 to the second catheter or the basket catheter 3402 in the present example.

In one example, the one or more of the actuator bosses 3456 or the one or more locking struts 3552 extend through the respective collar sidewall 3720 or collet sidewall 3730 shown in FIG. 37B. For instance, in one example, the actuator bosses 3456, as previously described, are separated from the remainder of the actuator collar 3454 with one or more slits 3722. In a similar manner, the locking strut 3552 of the locking collet 3550 is, in one example, separated from the remainder of the collet 3550 by one or more slits 3704. As shown in FIG. 37B as well as FIG. 37C, each of the actuator boss 3556 and the locking strut 3552 extend through the respective sidewalls 3720, 3730. For instance, in one example, the slits 3722 and slit 3704 extend through the sidewalls to permit deflection of portions of the actuator collar 3454 such as the actuator bosses 3456 or locking struts 3552 of the locking collet 3550.

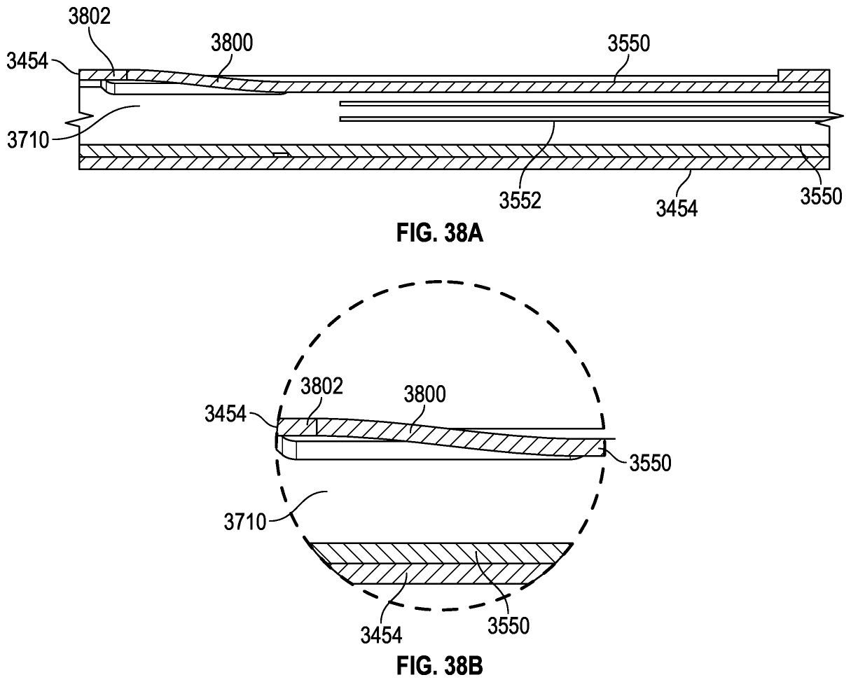

FIGS. 38A and 38B show an example of a detent system that maintains coupling between the actuator collar 3454 and the locking collet 3550 of the locking mechanism 3450. In this example, the locking collet 3550 is shown in cross-section and includes one or more collet detents 3800. In one example, the collet detent 3800 is a boss, flange, tine or the like that is plastically deformed or deflected into a radially extending configuration, shown in FIGS. 38A, B at an angle relative to the remainder of the locking collet 3550. Conversely, the actuator collar 3454 includes a collar shoulder 3802. The collar shoulder 3802 and collet detent 3800 are aligned. For instance, during assembly of the locking mechanism 3450, the actuator collar 3454 is slid over the locking collet 3550 in a proximal to distal manner. The actuator collar 3454 is moved distally until the collar shoulder 3802 passes the collet detent 3800 and the collet detent is freed to deflect outwardly into an intercepting configuration with the collar shoulder 3802. In this configuration, the actuator collar 3454 is now slidably coupled and retained along the locking collet 3550. For instance, proximal movement of the actuator collar 3454 relative to the position shown in FIG. 38A engages the collar shoulder 3802 with the collet detent 3800 and prevents further proximal movement of the actuator collar 3454.

A detailed view of the collet detent 3800 and collar shoulder 3802 is shown in FIG. 38B. For instance, the collet detent 3800 is shown in an upward deflected configuration and has an intercepting relationship with the collar shoulder 3802. Accordingly, proximal movement of the actuator collar 3454 is prevented by way of the engagement between the collar shoulder 3802 and the collar detent 3800.

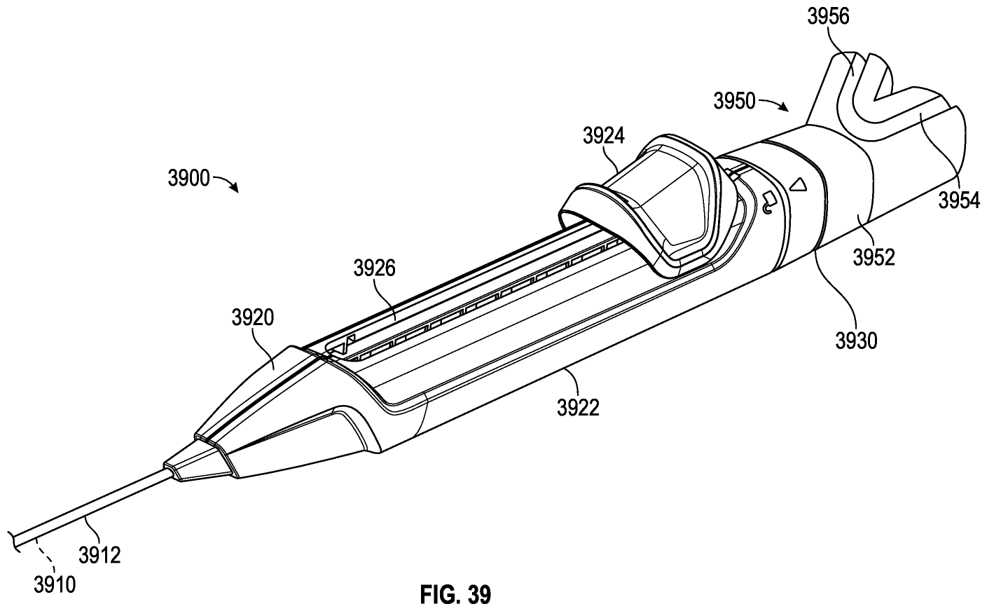

FIG. 39 shows a portion of a thrombectomy assembly 3900 including a manipulation controller 3920 coupled with a retrieval catheter such as a capture sleeve positioning tube 3910 coupled with a capture sleeve 114 (see FIG. 9) and a capture sleeve sheath 3912). In this example, the manipulation controller 3920 includes a sheath actuator 3924 in a retracted or first position relative to the remainder of the manipulation controller 3920 including the controller housing 3922. The sheath actuator 3924 is coupled with the capture sleeve sheath 3912. In FIG. 39, the sheath actuator 3924 is withdrawn or retracted along an actuator track 3926. In this configuration, with the sheath actuator 3924 withdrawn the capture sleeve sheath 3912 of the retrieval catheter is retracted relative to the capture sleeve positioning tube 3910 and the associated capture sleeve 114. With the sheath 3912 retracted the capture sleeve 114 is permitted to expand, for instance as shown in FIG. 9.

As described herein, the sheath actuator 3924 is, in one example, coupled with the capture sleeve sheath 3912 and permits movement of the sheath 3912 relative to the controller housing 3922. The capture sleeve positioning tube 3910, having the capture sleeve 114, is coupled with a different component of the controller housing 3922, for instance, a portion of a catheter lock 3930, a locking mechanism 3950 (described herein) or the like. Movement of the sheath actuator 3924 accordingly moves the capture sleeve sheath 3912 relative to the capture sleeve positioning tube 3910 and the capture sleeve 114 thereby deploying and retracting the capture sleeve 114. For instance, the proximal positioning of the sheath actuator 3924 correspondingly retracts the capture sleeve sheath 3912 and permits deployment of the capture sleeve 114, and the distal positioning of the sheath actuator distally moves the capture sleeve sheath 3912 and compresses the capture sleeve 114 (as well as filters, thrombus or the like therein).

Another example of a locking mechanism 3950 is shown in FIG. 39. In this example, the locking mechanism 3950 includes a lock body 3952 coupled with a component of the thrombectomy assembly 3900. For instance, the lock body 3952 is coupled with the capture sleeve positioning tube 3910, capture sleeve sheath 3912, the manipulation controller 3920 or the like. In the example shown in FIG. 39 the locking mechanism 3950 is coupled with the manipulation controller 3920.

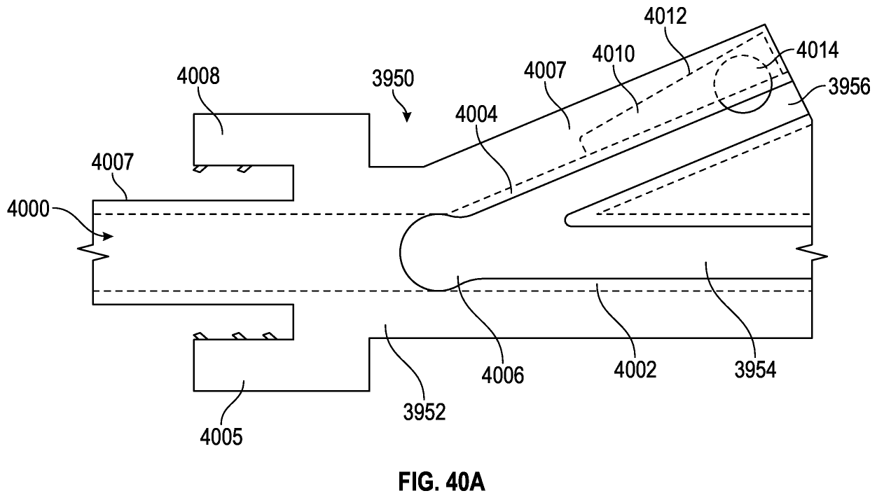

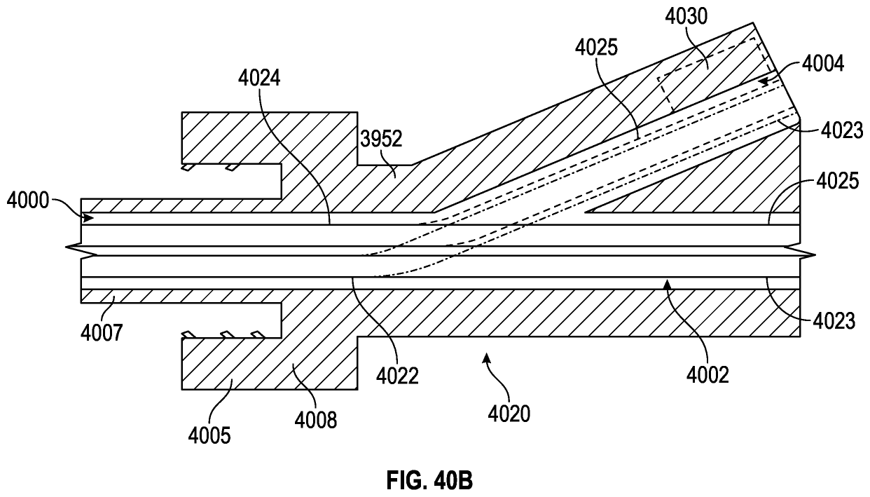

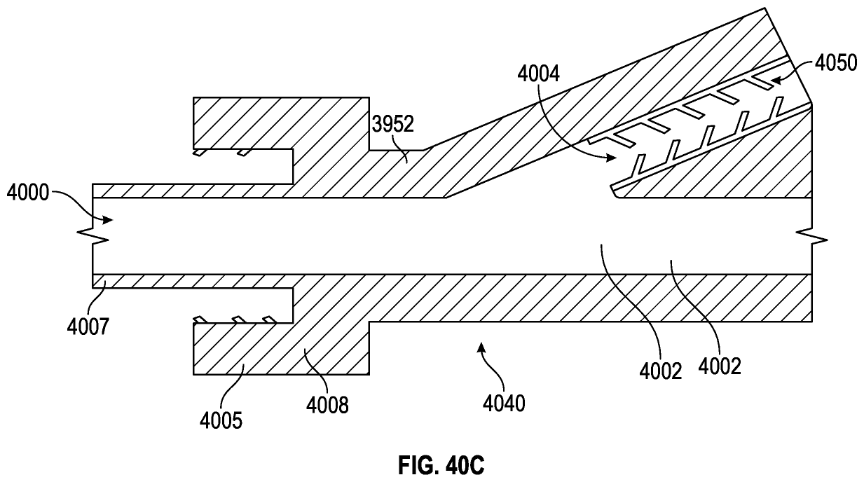

Referring again to FIG. 39, the locking mechanism 3950 includes a system access cleft 3954 and a lock access cleft 3956 that permit lateral access to instruments positioned in channels extend along the clefts. The clefts 3954, 3956 and their associated channels each communicate with a system lumen 4000 shown in FIG. 40A that extends through the lock body 3952. As described herein, the lock access cleft 3956 selectively receives one or more instruments and locks the one or more instruments in place relative to the locking mechanism 3950. The locking of the one or more instruments statically couples the instruments to instruments coupled with the locking mechanism, such as the manipulation controller 3920, retrieval catheter or the like. The static coupling permits unified movement of these components. In one example, the unified movement of these components (e.g., the manipulation controller and locked instruments) is relative to other instruments or components received in lumens of the manipulation controller and the system lumen 4000 of the locking mechanism 3950, thereby permitting selective relative sliding movement.

FIG. 40A is top view of the locking mechanism 3950 previously shown in FIG. 39. The system lumen 4000 is shown extending through the lock body 3952. In this example, the system lumen 4000 extends from a Luer lock 4008 or similar fitting (e.g., configured to couple directly or indirectly with an instrument such as a retrieval catheter) to an access intersection 4006. Each of the system access cleft 3954 and the lock access cleft 3956 extend from the access intersection 4006. For instance, the clefts 3954, 3956 are grooves, slits or the like extending through the lock body 3952 that provide access to associated channels.

The locking mechanism 3950 further includes a system channel 4002 extending from the system lumen 4000. In an example, the system channel 4002 is aligned with the system lumen 4000 (e.g., parallel, near parallel such as five degrees or the like). The system channel 4002 is laterally open. As shown in FIG. 40A the system access cleft 3954 extends along the system channel 4002 and thereby opens the channel 4002 for lateral access to instruments therein.

Additionally, the locking mechanism 3950 includes a locking channel 4004 extending from the system lumen 4000. In the example shown in FIG. 40A the locking channel 4004 is at an angle relative to the system lumen 4000 including, but not limited to, 15, 20, 30 degrees or the like. The locking channel 4004 is also laterally open, and includes the lock access cleft 3956 extending along the channel 4004.

The lock access cleft 3956 and the system access cleft 3954 each provide lateral access to the respective locking channel 4004 or the system channel 4002. As described herein, instruments such as catheters, guidewires or the like, positioned in the channels 4002, 4004 are readily accessed and moved between the channels 4002, 4004. For instance, a clinician may grasp instruments, such as a first catheter, second catheter, both or the like and laterally move the one or more catheters from the system channel 4002 through the system access cleft 3954 to the locking channel 4004 through the lock access cleft 3956. Optionally, the catheters are pivoted at the access intersection 4006 while moved between the channels 4002, 4004.

Positioning of an instrument (including instruments) in the locking channel 4004 through the lock access cleft 3956 positions the instrument in proximity to a lock element 4010 provided in the locking channel 4004. The lock element 4010 couples with the instrument and statically couples the instrument with the locking mechanism 3950. In one example, the lock body 3952 is itself coupled with an instrument, such as a retrieval catheter. With the first instrument, such as a catheter, in the locking channel 4004 the first instrument is statically coupled with the retrieval catheter, and movement of either of those components or the lock body 3952 is unified. Conversely, an instrument (e.g., a second catheter) positioned with the system lumen 4000 and system channel 4002 is permitted to move relative to the lock body 3952 and the first catheter statically coupled within the locking channel 4004. In the example, the lock body 3952, first catheter in the locking channel 4004 and the retrieval catheter are movable in a unitary manner relative to the second catheter in the system lumen 4000 and the system channel 4002. Accordingly, a second catheter such as a guidewire, positioning wire or the like remains in place while the remainder of the thrombectomy system is moved in a coordinated and unified manner (e.g., proximally, distally, rotated or the like). For instance, these components slide along the second catheter within the system lumen 4000 and the system channel 4002.

Referring again to FIG. 40A, one example of a lock element 4010 is shown. In this example, the lock element 4010 includes a wedge brake. The wedge brake includes a tapered track 4012 and a wedge 4014. In one example, the wedge 4014 includes a ball that moves along the tapered track 4012. In another example, the wedge 4014 includes a collet, shuttle or the like movable along the tapered track 4012. In operation, as the lock body 3952 is moved, for instance proximally, initial relative movement between a catheter within the locking channel 4004 and the wedge 4014 causes friction and moves the wedge 4014 along the tapered track 4012, for instance distally. The taper of the track 4012 engages the wedge 4014 between the track 4012 and the catheter, and the lock element 4010 accordingly statically couples the catheter to the lock body 3952. Conversely, in another example proximal movement of the catheter caused by pulling on the catheter relative to the lock body 3952 frictionally moves the wedge 4014 proximally, disengages the wedge 4014 from the track 4012 and thereby frees the catheter to move. In another example, the clinician grasps the catheter (e.g., extending out of the lock body 3952 from the locking channel 4004) and laterally moves the catheter through the lock access cleft 3956 out of the locking channel 4004 thereby disengaging the lock element 4010.

Referring now to FIG. 40B, an example of the thrombectomy assembly 3900 in operation is shown with another example of a locking mechanism 4020. The locking mechanism 4020 is sectioned. The system lumen 4000 includes first and second catheters 4022, 4024. First and second catheter portions 4023, 4025 of the first and second catheters 4022, 4024 are shown in solid and dashed lines indicative of selective placement of the portions in either of the system channel 4002 or locking channel 4004, respectively.

The locking mechanism 4020 includes another example of a lock element 4030. The lock element 4030 in this example includes a bias element, such as a magnet, spring, biased detent or the like configured to statically couple with either or both of the first or second catheter portions 4023, 4025 positioned with the locking channel 4004. In a similar manner to the lock element 4010 (a wedge brake) in FIG. 40A, the lock element 4030 couples with portions of one or more of the first or second catheters 4022, 4024. For instance, a magnetic lock element 4030 statically couples ferrous components of the first or second catheter portions 4023, 4025. In another example, a biased detent, spring or the like as the lock element 4030 mechanically engages with one or more of the first or second catheter portions 4023, 4025 to statically couple one or more of the catheters 4022, 4024 with the locking mechanism 4020.

In operation, the first and second catheters 4022, 4024 extend through the system lumen 4000 and the system channel 4002 in an unlocked configuration. The locking mechanism 4020 and components coupled to the mechanism, such as the manipulation controller 3920, retrieval catheter (such as the capture sleeve sheath 3912 and capture sleeve 114) are movable relative to the first and second catheters 4022, 4024. For instance, proximal or distal movement of the locking mechanism 4020 is relative to the catheters 4022, 4024. Additionally, the catheters 4022, 4024 are each movable relative to the other catheter 4024, 4022 and the locking mechanism 4020.

In a locked configuration one or more of the first or second catheters 4022, 4024 is moved from the system channel 4002 to the locking channel 4004. In one example, a second catheter portion 4025 of the second catheter 4024 is positioned in the locking channel 4004 while the first catheter portion 4023 of the first catheter 4022 remains positioned in the system channel 4002. For instance, the second catheter portion 4025 is grasped by a clinician laterally moved through the system access cleft 3954 (see FIG. 40A) and positioned into the locking channel 4044 through the lock access cleft 3956. The second catheter portion 4025 is isolated from the first catheter 4022, including the first catheter portion 4023. Both of the first and second catheters 4022, 4024 extend through the system lumen 4000 of the locking mechanism 4020.

With the second catheter portion 4025 (shown with dashed lines in Figure positioned in the locking channel 4044 the lock element 4030 statically couples the second catheter 4024 with the locking mechanism 4020, such as the lock body 3952. For example, the lock element 4030 magnetically couples the second catheter portion 4025 in place. In the locked configuration each of the second catheter 4024, the lock body 3952 (of the locking mechanism 4020) and components optionally coupled with the lock body like the manipulation controller 3920 are movable in a unified manner. These components are movable relative to the first catheter 4022 having the first catheter portion 4023 positioned in the system channel 4002 in contrast to the locking channel 4004. Accordingly, one or more of proximal, distal, rotational movement or the like of the second catheter 4024, lock body 3952 and associated components is relative to the first catheter 4022. In one example, the first catheter 4022 includes a procedural guidewire, and the procedural guide wire is used as a rail for slidable movement of the components held together in the locked configuration using the locking mechanism 4020. Alternatively, the first catheter 4022 is statically coupled within the locking channel 4004 and the second catheter 4024 is relatively movable.

In another example, each of the first and second catheter portions 4023, 4025 are positioned through the lock access cleft 4004 into the locking channel 3956. In this example of the locked configuration each of the first and second catheters 4022, 4024 are statically coupled with the locking mechanism 4020 with the lock element 4030. Accordingly, movement of the lock body 3952 or one or both of the catheters 4022, 4024 is transmitted to the other components and the locking mechanism 4020 and the catheters 4022, 4024 are moved in a unified manner.

If transition from the locked configuration to the unlocked configuration is specified the locked catheter 4024 (or catheters 4022, 4024) is moved from the locking channel 4004 through the lock access cleft 3956 to the system channel 3954, for instance through the system access cleft 3954. Movement from the locking channel 4004 disengages the static coupling of the lock element 4030 and permits relative movement between the catheters 4022, 4024 and the locking mechanism 4020.

FIG. 40C is another example of a locking mechanism 4040 including a lock element 4050 in the locking channel 4004. In this example, the lock element 4050 includes one or more cleats that engage with one or more of the first or second catheter portions 4023, 4025 to statically couple the portions with the lock body 3952. The cleats include, but are not limited to, brushes, barbs, knurling or the like. In another example, the cleats include butyl rubber or similar to provide a frictional or tacky interface between the lock element 4050 and a catheter position with the locking channel 4004. Optionally, the cleats are directed preferentially to initiate static coupling with directional movement. For instance, as shown in FIG. 40C, the cleats are directed proximally (e.g., have a proximally directed angle). Accordingly, proximal movement affirmatively engages the cleats against one or more catheter portions 4023, 4025 within the locking channel 4004 and thereby enhances the static coupling of the catheters with the lock body 3952.

In operation, the locking mechanism 4040 is transitioned between unlocked and locked configurations in a similar manner to other example mechanisms discussed herein. In the locked configuration a catheter portion, such as one or both of the first or second catheter portions 4023, 4025 is positioned through a lock access cleft, such as the cleft 3956 shown in FIG. 40A, and into the locking channel 4004. The lock element 4050 couples along one or more of the first or second catheter portions 4023, 4025 and statically couples the first or second catheters 4022, 4024 to the lock body 3952 of the locking mechanism 4040. Conversely, withdrawal of the first or second catheter portions 4023, 4025 from the locking channel 4004 (e.g., through the lock access cleft 3956) unlocks the one or more catheters 4022, 4024. In an example, the unlocked catheter portions 4023, 4025 are positioned through the system access cleft 3954 into the system channel 4002. The system channel 4002 is aligned with the system lumen 4000 to facilitate movement of the lock body 3952 relative to the catheters positioned in the system channel 4002 and system lumen 4000.

In another example, the lock element of the locking mechanism (e.g., 3950, 4020, 4040) includes a portion of the lock body 3952 that binds the first or second catheter portions 4023, 4025 that are deflected by the body 3952. For instance, referring to FIG. 40B, the first and second catheter portions 4023, 4025 are deflected while positioned in the locking channel 4004. In one example, lateral movement of one or more of the catheter portions from the system channel 4002 to the locking channel 4004 deflects the catheter portions as shown. For instance, the catheter portions are pivoted and deflected at the access intersection 4006. The lock body 3952, such as the angled walls of the locking channel 4004, in contrast to the walls of the system lumen 4000, engage with the deflected catheter portions. The engagement between the walls and the deflected catheter portions generates a normal force, and in combination with friction, the deflected catheter portion (e.g., 4023, 4025) is statically coupled with the lock body 3952. The frictional coupling with the locking channel 4004 (including its walls) is another example of a lock element that statically couples the one or more catheter portions with the lock body 3952 through angular binding of the catheter portions. Optionally, one or more curves, corners, angles, ridges or the like for the catheter portions to deflect around are provided with the locking channel 4004 to further enhance the static coupling.

To transition to the unlocked configuration the one or more catheter portions are moved from the locking channel 4004 thereby disengaging the frictional coupling. The catheter portions are relaxed from the deflected orientations shown in dashed lines in FIG. 40B and optionally positioned within the system channel 4002 (e.g., through the system access cleft 3954) to thereby permit relative movement between the locking mechanism and the catheter (or catheters 4022, 4024) positioned within the system channel 4002.

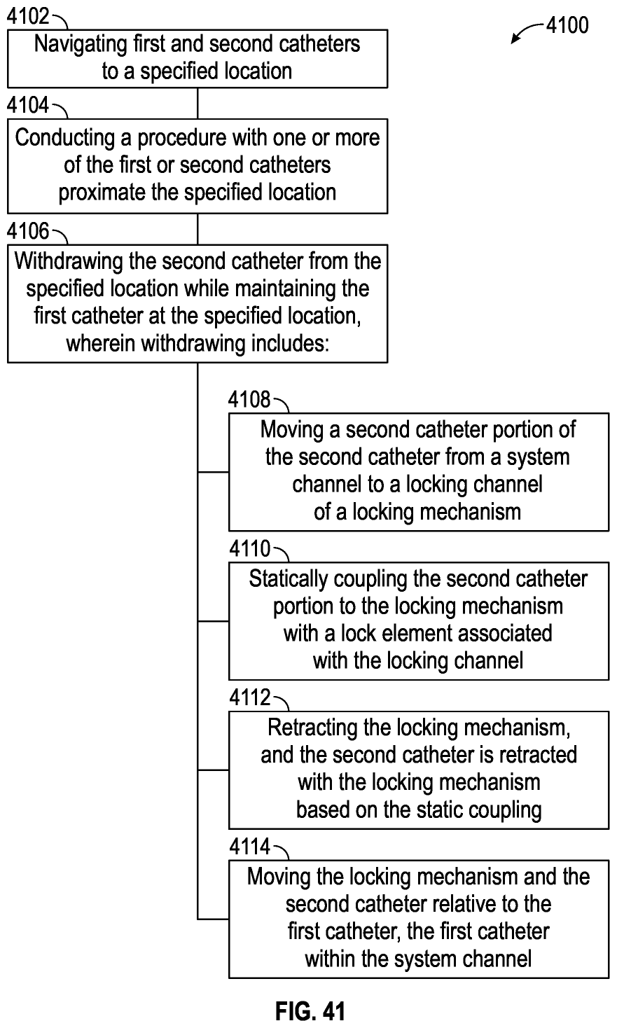

FIG. 41 shows one example of a method 4100 for locking one or more catheters. In describing the method 4100, reference is made to one or more components, features, functions, steps or the like previously described herein. Where convenient, reference is made to the components, features, functions, steps or the like with reference numerals. Reference numerals provided are exemplary and are not exclusive. For instance, components, features, functions, steps or the like described in the method 4100 include, but are not limited to, corresponding numbered elements provided herein, other corresponding features described herein (both numbered and unnumbered) as well as their equivalents.

At 4102 first and second catheters 4022, 4024 are navigated to a specified location. For example, one or more tools, elements or the like including filters, aspiration portions, sleeves or sleeves are positioned proximate to a treatment location having thrombus. At 4104 a procedure is conducted with one or more of the first or second catheters 4022, 4024 proximate the specified location. In one example, a thrombectomy procedure is conducted to capture and remove thrombus from the specified location.

At 4106 the second catheter 4024 is withdrawn from the specified location while the first catheter 4022, such as a guidewire, is maintained at the specified location. The first catheter 4022 is retained in position to optionally permit the conduct of additional procedures. Withdrawing of the second catheter 4024 includes at 4108 moving a second catheter portion 4025 of the second catheter 4024 from a system channel 4002 to a locking channel 4004 of a locking mechanism, such as (but not limited to) the locking mechanisms 3950, 4020, 4040. At 4110, the method 4100 includes statically coupling the second catheter portion 4025 to the locking mechanism with a lock element associated with the locking channel 4004. The lock element includes, but is not limited to, one or more a wedge brake, cleats, biasing element, angled binding of the catheter portion 4025 with the walls of the locking channel 4004 and system lumen 4000 or the like. The locking mechanism (e.g., 3950, 4020, 4040) is retracted, and the second catheter 4024 is retracted with the locking mechanism based on the static coupling. The locking mechanism is moved with the second catheter (e.g., moved as a consolidated assembly, unified movement or the like) relative to the first catheter 4022. In one example, the first catheter 4022 is positioned within the system channel 4002 to facilitate relative movement.

Several options for the method 4100 follow. In one example, withdrawing the second catheter from the specified location while maintaining the first catheter at the specified location includes isolating the first catheter 4022 in the system channel 4002 from the second catheter portion 4025 in the locking channel 4004. In another example, moving the second catheter portion 4025 to the locking channel 4004 includes laterally positioning the second catheter portion 4025 into the locking channel 4004 through a lock access cleft 3956 extending along the locking channel 4004. Optionally, moving the second catheter portion 4025 from the system channel 4002 includes laterally positioning the second catheter portion 4025 through a system access cleft 3954 extending along the system channel 4002.

In one example, moving the second catheter portion 4025 from the system channel 4002 to the locking channel 4004 includes pivoting the second catheter portion 4025 from the system channel 4002 to the locking channel 4004. Optionally, pivoting the second catheter portion 4025 includes pivoting the second catheter portion 4025 at an access intersection 4006 interconnecting the locking channel 4004 and the system channel 4002.

In another example, the method 4100 includes loading the first and second catheters 4022, 4024 into the locking mechanism through a system lumen 4000. The system lumen 4000 in communication with each of the system channel 4002 and the locking channel 4004.

### Various Notes and Aspects

Aspect 1 can include subject matter such as a locking catheter system comprising: a first catheter; a second catheter movably coupled with the first catheter; and a locking mechanism coupled with the first and second catheters, the locking mechanism includes: a lock body; a system lumen extending through the lock body, wherein the first and second catheters are received in the system lumen; a system channel extending from the system lumen, the system channel configured to receive one or more of the first or second catheters; a locking channel extending from the system lumen, the locking channel configured to receive one or more of the first or second catheters; and a lock element coupled with the locking channel, the lock element configured to statically couple one or more of the first or second catheters in the locking channel to the lock body.

Aspect 2 can include, or can optionally be combined with the subject matter of Aspect 1, to optionally include wherein the system lumen extends between a proximal open end and a distal open end, and the system lumen is enclosed between the proximal and distal open ends.

Aspect 3 can include, or can optionally be combined with the subject matter of one or any combination of Aspects 1 or 2 to optionally include wherein the lock body includes a system access cleft extending along the system channel; wherein the lock body includes a lock access cleft extending along the locking channel; and at least one of the first or second catheters is selectively positioned through each of the system access cleft or the lock access cleft into the respective system or locking channels.

Aspect 4 can include, or can optionally be combined with the subject matter of one or any combination of Aspects 1-3 to optionally include wherein the lock access cleft and the system access cleft includes cleft spacing, the locking channel includes a locking channel diameter and the system channel includes a system channel diameter, and the locking and system channel diameters are greater than the cleft spacing.

Aspect 5 can include, or can optionally be combined with the subject matter of one or any combination of Aspects 1-4 to optionally include wherein the lock body includes an access intersection: the system lumen extends to the access intersection; and the system channel and the locking channel extend from the access intersection

Aspect 6 can include, or can optionally be combined with the subject matter of Aspects 1-5 to optionally include wherein the lock element includes a wedge brake.

Aspect 7 can include, or can optionally be combined with the subject matter of Aspects 1-6 to optionally include wherein the wedge brake includes a tapered track and a wedge within the tapered track, the wedge in communication with the locking channel.

Aspect 8 can include, or can optionally be combined with the subject matter of Aspects 1-7 to optionally include wherein the wedge includes a ball.

Aspect 9 can include, or can optionally be combined with the subject matter of Aspects 1-8 to optionally include wherein the lock element includes one or more cleats.

Aspect 10 can include, or can optionally be combined with the subject matter of Aspects 1-9 to optionally include wherein the lock element includes one or more biasing elements.

Aspect 11 can include, or can optionally be combined with the subject matter of Aspects 1-10 to optionally include wherein the one or more biasing elements include one or more magnets.

Aspect 12 can include, or can optionally be combined with the subject matter of Aspects 1-11 to optionally include wherein the system channel is aligned with the system lumen; and the locking channel is angled relative to the system lumen.

Aspect 13 can include, or can optionally be combined with the subject matter of Aspects 1-12 to optionally include wherein the lock body includes an integral lock body.

Aspect 14 can include, or can optionally be combined with the subject matter of Aspects 1-13 to optionally include wherein the first and second catheters are longitudinally movable within the system channel relative to the lock body.

Aspect 15 can include, or can optionally be combined with the subject matter of Aspects 1-14 to optionally include wherein the first catheter includes a procedural guidewire, the second catheter includes a basket catheter; and in a locked configuration the procedural guidewire is received in the system channel, the basket catheter is received in the locking channel and statically coupled with the lock body, and movement of the lock body correspondingly moves the basket catheter relative to the procedural guidewire.

Aspect 16 can include, or can optionally be combined with the subject matter of Aspects 1-15 to optionally include a retrieval catheter coupled with the locking mechanism, the procedural guidewire and basket catheter extend through the retrieval catheter; and in the locked configuration movement of the lock body correspondingly moves the retrieval catheter and basket catheter relative to the procedural guidewire.

Aspect 17 can include, or can optionally be combined with the subject matter of Aspects 1-16 to optionally include a locking catheter system comprising: a first catheter; a second catheter; a locking mechanism coupled with the first and second catheters, the locking mechanism includes: a lock body having a system lumen extending through the lock body, wherein the first and second catheters are received in the system lumen; a system channel extending from the system lumen, the system channel configured to receive one or more of the first or second catheters; a locking channel extending from the system lumen, the locking channel including a lock element, the locking channel configured to receive one or more of the first or second catheters; and wherein the locking mechanism includes locked and unlocked configurations: in the unlocked configuration the first and second catheters are received in the system channel, and the first and second catheters are movable relative to the lock body; and in the locked configuration at least the second catheter is received in the locking channel, the second catheter is statically coupled with the lock body, and the first catheter is movable relative to the lock body.

Aspect 18 can include, or can optionally be combined with the subject matter of Aspects 1-17 to optionally include wherein movement of the lock body correspondingly moves the second catheter relative to the first catheter.

Aspect 19 can include, or can optionally be combined with the subject matter of Aspects 1-18 to optionally include wherein the first catheter includes a procedural guidewire, and the second catheter includes a basket catheter; and in the locked configuration movement of the lock body correspondingly moves the basket catheter relative to the procedural guidewire.

Aspect 20 can include, or can optionally be combined with the subject matter of Aspects 1-19 to optionally include a retrieval catheter coupled with the locking mechanism, the procedural guidewire and basket catheter extend through the retrieval catheter; and in the locked configuration movement of the lock body correspondingly moves the retrieval catheter and basket catheter relative to the procedural guidewire.

Aspect 21 can include, or can optionally be combined with the subject matter of Aspects 1-20 to optionally include wherein the system lumen extends between a proximal open end and a distal open end, and the system lumen is enclosed between the proximal and distal open ends.

Aspect 22 can include, or can optionally be combined with the subject matter of Aspects 1-21 to optionally include wherein the lock body includes a system access cleft extending along the system lumen; wherein the lock body includes a lock access cleft extending along the locking channel; and in the locked configuration the second catheter is selectively positioned through the lock access cleft into the locking channel.

Aspect 23 can include, or can optionally be combined with the subject matter of Aspects 1-22 to optionally include wherein the lock access cleft and the system access cleft includes cleft spacing, the locking channel includes a locking channel diameter and the system channel includes a system channel diameter, and the locking and system channel diameters are greater than the cleft spacing.

Aspect 24 can include, or can optionally be combined with the subject matter of Aspects 1-23 to optionally include wherein the lock element includes a wedge brake.

Aspect 25 can include, or can optionally be combined with the subject matter of Aspects 1-24 to optionally include wherein the lock element includes one or more cleats.

Aspect 26 can include, or can optionally be combined with the subject matter of Aspects 1-25 to optionally include wherein the lock element includes one or more biasing elements.

Aspect 27 can include, or can optionally be combined with the subject matter of Aspects 1-26 to optionally include wherein the system channel is aligned with the system lumen; and the locking channel is angled relative to the system lumen.

Aspect 28 can include, or can optionally be combined with the subject matter of Aspects 1-27 to optionally include a method of locking one or more catheters comprising: navigating first and second catheters to a specified location; conducting a procedure with one or more of the first or second catheters proximate the specified location; and withdrawing the second catheter from the specified location while maintaining the first catheter at the specified location, wherein withdrawing includes: moving a second catheter portion of the second catheter from a system channel to a locking channel of a locking mechanism; statically coupling the second catheter portion to the locking mechanism with a lock element associated with the locking channel; retracting the locking mechanism, and the second catheter is retracted with the locking mechanism based on the static coupling; moving the locking mechanism and the second catheter relative to the first catheter, the first catheter within the system channel.

Aspect 29 can include, or can optionally be combined with the subject matter of Aspects 1-28 to optionally include wherein withdrawing the second catheter from the specified location while maintaining the first catheter at the specified location includes isolating the first catheter in the system channel from the second catheter portion in the locking channel.

Aspect 30 can include, or can optionally be combined with the subject matter of Aspects 1-29 to optionally include wherein moving the second catheter portion to the locking channel includes laterally positioning the second catheter portion into the locking channel through a lock access cleft extending along the locking channel.

Aspect 31 can include, or can optionally be combined with the subject matter of Aspects 1-30 to optionally include wherein moving the second catheter portion from the system channel includes laterally positioning the second catheter portion through a system access cleft extending along the system channel.

Aspect 32 can include, or can optionally be combined with the subject matter of Aspects 1-31 to optionally include wherein moving the second catheter portion from the system channel to the locking channel includes pivoting the second catheter portion from the system channel to the locking channel.

Aspect 33 can include, or can optionally be combined with the subject matter of Aspects 1-32 to optionally include wherein pivoting the second catheter portion includes pivoting the second catheter portion at an access intersection interconnecting the locking channel and the system channel.

Aspect 34 can include, or can optionally be combined with the subject matter of Aspects 1-33 to optionally include loading the first and second catheters into the locking mechanism through a system lumen, the system lumen in communication with each of the system channel and the locking channel.

Aspect 35 can include, or can optionally be combined with the subject matter of Aspects 1-34 to optionally include wherein the lock element includes one or more of a wedge brake, a cleat, a biasing element or angled binding.

Aspect 36 can include, or can optionally be combined with the subject matter of Aspects 1-35 to optionally include a locking catheter system comprising: a basket catheter having one or more baskets proximate a basket catheter distal end, the basket catheter includes a basket catheter lumen; an inner catheter received in the basket catheter lumen; and a locking mechanism coupled with the basket catheter assembly, the locking mechanism includes: a locking collet coupled with the basket catheter, the locking collet includes one or more locking struts; an actuator collar slidably coupled along the locking collet, the actuator collar includes one or more actuator bosses; and wherein in a locked configuration the one or more actuator bosses deflect the one or more locking struts, and the deflected one or more locking struts engage with the inner catheter and hold the basket catheter static relative to the inner catheter.

Aspect 37 can include, or can optionally be combined with the subject matter of Aspects 1-36 to optionally include wherein the inner catheter includes one or more of a guidewire or a catheter having an inner catheter lumen.

Aspect 38 can include, or can optionally be combined with the subject matter of Aspects 1-37 to optionally include wherein the basket catheter assembly includes a basket sheath slidably coupled along the basket catheter, the basket sheath is configured to selectively cover the one or more baskets.

Aspect 39 can include, or can optionally be combined with the subject matter of Aspects 1-38 to optionally include wherein the basket catheter includes a shoulder, and in the locked configuration the actuator collar is engaged with the shoulder.

Aspect 40 can include, or can optionally be combined with the subject matter of Aspects 1-39 to optionally include wherein the one or more actuator bosses include a plurality of actuator bosses along an inner surface of the actuator collar.

Aspect 41 can include, or can optionally be combined with the subject matter of Aspects 1-40 to optionally include wherein the plurality of actuator bosses are directed toward the locking collet.

Aspect 42 can include, or can optionally be combined with the subject matter of Aspects 1-41 to optionally include wherein the one or more locking struts include a plurality of locking struts, and the plurality of actuator bosses and the plurality of locking struts have coincident profiles in the locked configuration.

Aspect 43 can include, or can optionally be combined with the subject matter of Aspects 1-42 to optionally include wherein the coincident profiles include one of fully coincident or partially coincident profiles.

Aspect 44 can include, or can optionally be combined with the subject matter of Aspects 1-43 to optionally include wherein the one or more locking struts include a plurality of locking struts, and the plurality of actuator bosses and the plurality of locking struts have symmetric profiles.

Aspect 45 can include, or can optionally be combined with the subject matter of Aspects 1-44 to optionally include wherein at least one locking strut of the one or more locking struts includes a strut member and at least one strut end coupling the strut member with a remainder of the locking collet.

Aspect 46 can include, or can optionally be combined with the subject matter of Aspects 1-45 to optionally include wherein the at least one strut end includes a first strut end and a second strut end, and the strut member extends between the first and second strut ends.

Aspect 47 can include, or can optionally be combined with the subject matter of Aspects 1-46 to optionally include wherein the one or more locking struts is parallel to a longitudinal axis of the locking collet.

Aspect 48 can include, or can optionally be combined with the subject matter of Aspects 1-47 to optionally include wherein the locking collet includes a collet sidewall, and the one or more locking struts each include a strut member, and the strut member is separated from the collet sidewall with a slit extending through the collet sidewall.

Aspect 49 can include, or can optionally be combined with the subject matter of Aspects 1-48 to optionally include wherein the actuator collar includes a collar sidewall, and the one or more actuator bosses each include a flange, and flange is separated from the collar sidewall with a slit extending through the collar sidewall.

Aspect 50 can include, or can optionally be combined with the subject matter of Aspects 1-49 to optionally include wherein in an unlocked configuration the one or more actuator bosses are misaligned with the one or more locking struts and the basket catheter movable relative to the guidewire.

Aspect 51 can include, or can optionally be combined with the subject matter of Aspects 1-50 to optionally include wherein the basket catheter includes a catheter shoulder spaced from the actuator collar in an unlocked configuration, and in the locked configuration the actuator collar engages with the catheter shoulder.

Aspect 52 can include, or can optionally be combined with the subject matter of Aspects 1-51 to optionally include a locking catheter system comprising: a first catheter; a second catheter having a second catheter lumen, the first catheter is received in the second catheter lumen; and a locking mechanism coupled with the second catheter, the locking mechanism includes: a locking collet coupled with the second catheter, the locking collet includes one or more locking struts; an actuator collar slidably coupled along the locking collet, the actuator collar includes one or more actuator bosses; and wherein in a locked configuration the one or more actuator bosses deflect the one or more locking struts, and the deflected one or more locking struts engage with the first catheter and hold the second catheter static relative to the first catheter.

Aspect 53 can include, or can optionally be combined with the subject matter of Aspects 1-52 to optionally include wherein the first catheter includes one or more of a guidewire or a catheter having an inner catheter lumen.

Aspect 54 can include, or can optionally be combined with the subject matter of Aspects 1-53 to optionally include wherein the second catheter includes a basket catheter having one or more baskets proximate a basket catheter distal end.

Aspect 55 can include, or can optionally be combined with the subject matter of Aspects 1-54 to optionally include wherein the one or more actuator bosses include a plurality of actuator bosses along an inner surface of the actuator collar.

Aspect 56 can include, or can optionally be combined with the subject matter of Aspects 1-55 to optionally include wherein the one or more locking struts include a plurality of locking struts, and the plurality of actuator bosses and the plurality of locking struts have coincident profiles in the locked configuration.

Aspect 57 can include, or can optionally be combined with the subject matter of Aspects 1-56 to optionally include wherein at least one locking strut of the one or more locking struts includes a strut member and at least one strut end coupling the strut member with a remainder of the locking collet.

Aspect 58 can include, or can optionally be combined with the subject matter of Aspects 1-57 to optionally include wherein the locking collet includes a collet sidewall, and the one or more locking struts each include a strut member, and the strut member is separated from the collet sidewall with a slit extending through the collet sidewall.

Aspect 59 can include, or can optionally be combined with the subject matter of Aspects 1-58 to optionally include wherein the actuator collar includes a collar sidewall, and the one or more actuator bosses each include a flange, and flange is separated from the collar sidewall with a slit extending through the collar sidewall.

Aspect 60 can include, or can optionally be combined with the subject matter of Aspects 1-59 to optionally include wherein in an unlocked configuration the one or more actuator bosses are misaligned with the one or more locking struts and the basket catheter movable relative to the guidewire.

Aspect 61 can include, or can optionally be combined with the subject matter of Aspects 1-60 to optionally include a method of locking catheters comprising: navigating a second catheter over a first catheter, the first catheter received in a second catheter lumen of the second catheter; and securing the first and second catheters together with a locking mechanism, securing includes: moving an actuator collar along a locking collet; aligning the one or more actuator bosses of the actuator collar with one or more locking struts of the locking collet; deflecting the one or more locking struts with the aligned one or more actuator bosses; and wherein the deflected one or more locking struts engage with the first catheter and hold the second catheter static relative to the first catheter.

Aspect 62 can include, or can optionally be combined with the subject matter of Aspects 1-61 to optionally include navigating the first catheter distal to a specified location in vasculature.

Aspect 63 can include, or can optionally be combined with the subject matter of Aspects 1-62 to optionally include wherein navigating the second catheter includes navigating the second catheter over the first catheter distal to the specified location.

Aspect 64 can include, or can optionally be combined with the subject matter of Aspects 1-63 to optionally include deploying one or more baskets distal to the specified location.

Aspect 65 can include, or can optionally be combined with the subject matter of Aspects 1-64 to optionally include navigating a third catheter over the second catheter and the locking mechanism proximal to the specified location, wherein the second catheter and the locking mechanism are received in a third catheter lumen of the third catheter.

Aspect 66 can include, or can optionally be combined with the subject matter of Aspects 1-65 to optionally include retracting the third catheter over the second catheter and the locking mechanism; unsecuring the second catheter from the first catheter including misaligning the one or more actuator bosses relative to the one or more locking struts; and retracting the second catheter from the specified location over the first catheter.

Aspect 67 can include, or can optionally be combined with the subject matter of Aspects 1-66 to optionally include wherein aligning the one or more actuator bosses of the actuator collar with one or more locking struts includes aligning a plurality of actuator bosses with a plurality of locking struts.

Aspect 68 can include, or can optionally be combined with the subject matter of Aspects 1-67 to optionally include wherein aligning the plurality of actuator bosses with the plurality of locking struts includes aligning a profile of the plurality of actuator bosses to a matched profile of the plurality of locking struts.

Aspect 69 can include, or can optionally be combined with the subject matter of Aspects 1-68 to optionally include wherein moving the actuator collar along the locking collet includes one or more of longitudinal or rotational movement of the actuator collar relative to the locking collet.

Each of these non-limiting aspects can stand on its own, or can be combined in various permutations or combinations with one or more of the other aspects.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A locking catheter system comprising:
a first catheter;
a second catheter movably coupled with the first catheter; and
a locking mechanism coupled with the first and second catheters, the locking mechanism includes:
a lock body;
a system lumen extending through the lock body, wherein the first and second catheters are received in the system lumen;
a system channel extending from the system lumen, the system channel configured to receive one or more of the first or second catheters;
a locking channel extending from the system lumen, the locking channel configured to receive one or more of the first or second catheters; and
a lock element coupled with the locking channel, the lock element configured to statically couple one or more of the first or second catheters in the locking channel to the lock body.

2. The locking catheter system of claim 1, wherein the system lumen extends between a proximal open end and a distal open end, and the system lumen is enclosed between the proximal and distal open ends.

3. The locking catheter system of claim 1, wherein the lock body includes a system access cleft extending along the system channel;
wherein the lock body includes a lock access cleft extending along the locking channel; and
at least one of the first or second catheters is selectively positioned through each of the system access cleft or the lock access cleft into the respective system or locking channels.

4. The locking catheter system of claim 3, wherein the lock access cleft and the system access cleft includes cleft spacing, the locking channel includes a locking channel diameter and the system channel includes a system channel diameter, and the locking and system channel diameters are greater than the cleft spacing.

5. The locking catheter system of claim 3, wherein the lock body includes an access intersection:
the system lumen extends to the access intersection; and
the system channel and the locking channel extend from the access intersection

6. The locking catheter system of claim 1, wherein the lock element includes a wedge brake.

7. The locking catheter system of claim 6, wherein the wedge brake includes a tapered track and a wedge within the tapered track, the wedge in communication with the locking channel.

8. The locking catheter system of claim 7, wherein the wedge includes a ball.

9. The locking catheter system of claim 1, wherein the lock element includes one or more cleats.

10. The locking catheter system of claim 1, wherein the lock element includes one or more biasing elements.

11. The locking catheter system of claim 10, wherein the one or more biasing elements include one or more magnets.

12. The locking catheter system of claim 1, wherein the system channel is aligned with the system lumen; and the locking channel is angled relative to the system lumen.

13. The locking catheter system of claim 1, wherein the lock body includes an integral lock body.

14. The locking catheter system of claim 1, wherein the first and second catheters are longitudinally movable within the system channel relative to the lock body.

15. The locking catheter system of claim 1, wherein the first catheter includes a procedural guidewire, the second catheter includes a basket catheter; and
in a locked configuration the procedural guidewire is received in the system channel, the basket catheter is received in the locking channel and statically coupled with the lock body, and movement of the lock body correspondingly moves the basket catheter relative to the procedural guidewire.

16. The locking catheter system of claim 15 comprising a retrieval catheter coupled with the locking mechanism, the procedural guidewire and basket catheter extend through the retrieval catheter; and
in the locked configuration movement of the lock body correspondingly moves the retrieval catheter and basket catheter relative to the procedural guidewire.

17. A locking catheter system comprising:
a first catheter;
a second catheter;
a locking mechanism coupled with the first and second catheters, the locking mechanism includes:
a lock body having a system lumen extending through the lock body, wherein the first and second catheters are received in the system lumen;
a system channel extending from the system lumen, the system channel configured to receive one or more of the first or second catheters;
a locking channel extending from the system lumen, the locking channel including a lock element, the locking channel configured to receive one or more of the first or second catheters; and
wherein the locking mechanism includes locked and unlocked configurations:
in the unlocked configuration the first and second catheters are received in the system channel, and the first and second catheters are movable relative to the lock body; and
in the locked configuration at least the second catheter is received in the locking channel, the second catheter is statically coupled with the lock body, and the first catheter is movable relative to the lock body.

18. The locking catheter system of claim 17, wherein movement of the lock body correspondingly moves the second catheter relative to the first catheter.

19. The locking catheter system of claim 17, wherein the first catheter includes a procedural guidewire, and the second catheter includes a basket catheter; and
in the locked configuration movement of the lock body correspondingly moves the basket catheter relative to the procedural guidewire.

20. The locking catheter system of claim 19 comprising a retrieval catheter coupled with the locking mechanism, the procedural guidewire and basket catheter extend through the retrieval catheter; and
in the locked configuration movement of the lock body correspondingly moves the retrieval catheter and basket catheter relative to the procedural guidewire.

21. The locking catheter system of claim 17, wherein the system lumen extends between a proximal open end and a distal open end, and the system lumen is enclosed between the proximal and distal open ends.

22. The locking catheter system of claim 17, wherein the lock body includes a system access cleft extending along the system lumen;
wherein the lock body includes a lock access cleft extending along the locking channel; and
in the locked configuration the second catheter is selectively positioned through the lock access cleft into the locking channel.

23. The locking catheter system of claim 22, wherein the lock access cleft and the system access cleft includes cleft spacing, the locking channel includes a locking channel diameter and the system channel includes a system channel diameter, and the locking and system channel diameters are greater than the cleft spacing.

24. The locking catheter system of claim 17, wherein the lock element includes a wedge brake.

25. The locking catheter system of claim 17, wherein the lock element includes one or more cleats.

26. The locking catheter system of claim 17, wherein the lock element includes one or more biasing elements.

27. The locking catheter system of claim 17, wherein the system channel is aligned with the system lumen; and the locking channel is angled relative to the system lumen.

28. A method of locking one or more catheters comprising:
navigating first and second catheters to a specified location;
conducting a procedure with one or more of the first or second catheters proximate the specified location; and
withdrawing the second catheter from the specified location while maintaining the first catheter at the specified location, wherein withdrawing includes:
moving a second catheter portion of the second catheter from a system channel to a locking channel of a locking mechanism;
statically coupling the second catheter portion to the locking mechanism with a lock element associated with the locking channel;
retracting the locking mechanism, and the second catheter is retracted with the locking mechanism based on the static coupling;
moving the locking mechanism and the second catheter relative to the first catheter, the first catheter within the system channel.

29. The method of claim 28, wherein withdrawing the second catheter from the specified location while maintaining the first catheter at the specified location includes isolating the first catheter in the system channel from the second catheter portion in the locking channel.

30. The method of claim 28, wherein moving the second catheter portion to the locking channel includes laterally positioning the second catheter portion into the locking channel through a lock access cleft extending along the locking channel.

31. The method of claim 30, wherein moving the second catheter portion from the system channel includes laterally positioning the second catheter portion through a system access cleft extending along the system channel.

32. The method of claim 28, wherein moving the second catheter portion from the system channel to the locking channel includes pivoting the second catheter portion from the system channel to the locking channel.

33. The method of claim 32, wherein pivoting the second catheter portion includes pivoting the second catheter portion at an access intersection interconnecting the locking channel and the system channel.

34. The method of claim 28 comprising loading the first and second catheters into the locking mechanism through a system lumen, the system lumen in communication with each of the system channel and the locking channel.

35. The method of claim 28, wherein the lock element includes one or more of a wedge brake, a cleat, a biasing element or angled binding.
